(19) United States
(12) Patent Application Publication
(10) Pub. No.: US 2024/0034762 A1
(43) Pub. Date: Feb. 1, 2024

(54) Compositions and Methods For Inhibiting FGF23 Activity

(71) Applicant: YALE UNIVERSITY, New Haven, CT (US)

(72) Inventors: Joseph Schlessinger, New Haven, CT (US); Yoshihisa Suzuki, New Haven, CT (US); Irit Lax, New Haven, CT (US)

(21) Appl. No.: 18/043,894

(22) PCT Filed: Sep. 2, 2021
(86) PCT No.: PCT/US2021/048915

§ 371 Date: Mar. 2, 2023

Related U.S. Application Data
(60) Provisional application No. 63/074,267, filed on Sep. 3, 2020.

(51) Int. Cl. C07K 14/50; C12N 15/63; A61P 5/00

(52) U.S. Cl. CPC C07K 14/50; C12N 15/63; A61P 5/00; C07K 2319/30

(57) ABSTRACT

The present disclosure provides in one aspect a construct comprising the R2 region of FGF23. In other embodiments, the construct functions as a FGF23 antagonist by blocking FGF23 binding to α-Klotho and cell signaling via FGFR activation. In yet other embodiments, the construct prevents FGFR activation. In yet other embodiments, the construct of the present disclosure can be used to treat diseases or disorders related to FGF23 dysregulation and/or overexpression, such as but not limited to phosphate metabolism disorders.

### CROSS-REFERENCE TO RELATED APPLICATIONS

The present application claims priority under 35 U.S.C. § 119(e) to U.S. Provisional Patent Application No. 63/074,267, filed Sep. 3, 2020, which is incorporated herein by reference in its entirety.

### SEQUENCE LISTING

The ASCII text file named “047162-7298WO1(01447)_Seq_Listing_ST25” created on Sep. 2, 2021, comprising 41.5 Kbytes, is hereby incorporated by reference in its entirety.

### BACKGROUND OF THE INVENTION

The large family of fibroblast growth factors (FGFs) has important roles in regulating critical cellular processes during embryonic development and homeostasis of normal tissues. The majority of FGFs act as cytokines or hormone-like proteins that mediate their pleiotropic cellular processes by binding to cell surface receptors endowed with intrinsic tyrosine kinase activity (FGFRs). Most receptor tyrosine kinases (RTKs) are activated by a single ligand molecule that binds with high affinity to the extracellular domain of its cognate RTK, with a dissociation constant in the sub-nM range. In contrast, the binding affinities of FGFs to FGFRs are at least 1000-10,000 fold weaker with dissociation constants in the sub-μM range. The weak binding affinities towards FGFRs of the largest subfamily of FGF molecules designated canonical-FGFs are offset by interactions with cell surface heparan sulphate proteoglycans (HSPG). Both biochemical and structural studies revealed how multiple interactions between heparin or HSPG with both FGF and FGFR mediate tight association enabling robust receptor dimerization and tyrosine kinase activation.

The three members of endocrine-FGFs (FGF19, FGF21, and FGF23) represent an additional subfamily of FGF molecules. Endocrine-FGFs function as circulating hormones that play essential roles in the control of various metabolic processes. In addition to the conserved FGF-domain found in all FGF ligands, endocrine FGFs contain unique C-terminal tails composed of 46 amino acids (FGF19), 34 amino acids (FGF21), and 89 amino acids (FGF23) amino acids that serve as specific and high affinity ligands for the two members of Klotho family of surface receptors. α-Klotho serves as a high affinity receptor for FGF23, while β-Klotho functions as a high affinity surface receptor for both FGF19 and FGF21. Structural analyses of free and ligand occupied Klotho proteins revealed the molecular basis underlying the specificity and high affinity of α-Klotho and β-Klotho towards endocrine FGFs. Klotho proteins function as the primary receptors for endocrine FGFs whereas FGFR functions as a catalytic subunit that mediates cell signaling via its tyrosine kinase domain. Accordingly, endocrine-FGFs stimulate their cellular responses by forming a ternary complex with Klotho proteins and FGFRs to induce receptor dimerization, tyrosine kinase activation, and cell signaling. Unlike FGFRs that are ubiquitously expressed, the expression patterns of α-Klotho and β-Klotho are restricted to specific tissues and organs, thus enabling specific targeting of endocrine FGFs to stimulate their physiological responses in specific cells and tissues. The ability of endocrine FGFs to circulate is attributed to the loss of conserved heparin binding sites that are essential for the function of canonical FGFs.

FGF23 is a 32-kDa glycoprotein, mainly produced in the bone by osteoblasts and osteocytes, that serves as a key hormone, regulating phosphate homeostasis, vitamin D and calcium metabolism. Circulating levels of physiologically active FGF23 are regulated by proteolytic cleavage to produce an FGF23 molecule lacking its unique C-terminal tail. The cleavage resulting in FGF23 inactivation prevents assembly of the FGF23/FGFR/α-Klotho signaling complex resulting in FGF23 inactivation. Additionally, the processing of FGF23 includes several post translational modifications that affect its stability and susceptibility toward proteolysis. Secreted FGF23 was shown to be 0-glycosylated in its C-terminal cleavage site to protect the protein from C-terminal cleavage. In order for the cleavage site to be exposed, FGF23 has to be first phosphorylated in this region. Phosphorylation prevents glycosylation and exposes the cleavage site to proteolysis.

There is a need in the art to identify compositions and methods that can be used to modulate (e.g. inhibit or stimulate) the function and action of α-Klotho and the activity of FGF receptors and the signaling pathways activated by endocrine FGFs, such as but not limited to FGF23. In certain embodiments, these compositions and methods are useful in treating, ameliorating and/or preventing diseases (such as, but not limited to, metabolic diseases and/or cancer) associated with endocrine-FGFs, such as but not limited to FGF23. The present invention fulfills these needs.

### SUMMARY

In some embodiments, the instant specification is directed to, although not limited to, the follows:

Embodiment 1 provides a non-natural soluble construct including an amino acid sequence that is at least 90% identical to amino acids 212-239 of SEQ ID NO:5 or a biologically active fragment thereof.

Embodiment 2 provides the construct of embodiment 1, which includes amino acids 212-239 of SEQ ID NO:5 or a biologically active fragment thereof.

Embodiment 3 provides the construct of any of embodiments 1-2, which includes amino acids 212-239 of SEQ ID NO:5.

Embodiment 4 provides the construct of any of embodiments 1-3, which is fused to a stability enhancing domain.

Embodiment 5 provides the construct of embodiment 4, wherein the stability enhancing domain includes at least one of albumin, thioredoxin, glutathione S-transferase, and/or a Fc region of an antibody.

Embodiment 6 provides the construct of embodiment 5, wherein the Fc region is IgG Fc.

Embodiment 7 provides the construct of embodiment 6, wherein the Fc region is the Fc domain of human immunoglobulin 1 (IgG1), human immunoglobulin 2 (IgG2), human immunoglobulin 3 (IgG3), and/or human immunoglobulin 4 (IgG4).

Embodiment 8 provides the construct of any of embodiments 4-7, wherein the stability enhancing domain is fused with the N-terminus of the polypeptide.

Embodiment 9 provides the construct of any of embodiments 4-7, wherein the stability enhancing domain is fused with the C-terminus of the polypeptide.

Embodiment 10 provides the construct of any of embodiments 4-9, wherein the stability enhancing domain is directly fused to the polypeptide.

Embodiment 11 provides the construct of any of embodiments 4-10, wherein the stability enhancing domain is fused through a linker to the polypeptide.

Embodiment 12 provides the constrict of embodiment 11, wherein the linker includes about 1-18 amino acids and/or 1-20 (ethylene glycol and/or propylene glycol) units.

Embodiment 13 provides the construct of any of embodiments 11-12, wherein the C-terminus of the linker fused to the N-terminus of the polypeptide is not one of the following: APASCSQELP (SEQ ID NO:20), PASCSQELP (SEQ ID NO:21), ASCSQELP (SEQ ID NO:22), SCSQELP (SEQ ID NO:23), CSQELP (SEQ ID NO:24), SQELP (SEQ ID NO:25), QELP (SEQ ID NO:26), ELP, LP, P.

Embodiment 14 provides the construct of any of embodiments 11-12, wherein the N-terminus of the linker fused to the C-terminus of the polypeptide is not one of the following: GPEGCRPFAKF (SEQ ID NO:27), GPEGCRPFAK (SEQ ID NO:28), GPEGCRPFA (SEQ ID NO:29), GPEGCRPF (SEQ ID NO:30), GPEGCRP (SEQ ID NO:31), GPEGCR (SEQ ID NO:32), GPEGC (SEQ ID NO:33), GPEG (SEQ ID NO:34), GPE, GP, G.

Embodiment 15 provides the construct of any of embodiments 1-14, which is pegylated, at least partially methylated, and/or C-terminus amidated.

Embodiment 16 provides a nucleic acid sequence that encodes the construct of any of embodiments 1-15.

Embodiment 17 provides a vector including the nucleic acid sequence of embodiment 16.

Embodiment 18 provides the vector of embodiment 17, which is an expression vector.

Embodiment 19 provides the vector of any of embodiments 17-18, which is an autonomously replicating or an integrative mammalian cell vector.

Embodiment 20 provides a cell, cells, or a plurality of cells including the nucleic acid of embodiment 16 or the vector of any of embodiment 17-19.

Embodiment 21 provides a method of treating, ameliorating, and/or preventing an endocrine FGF-related disease or disorder in a mammal, the method including administering to the mammal a therapeutically effective amount of the construct of any of embodiments 1-Embodiment 22 provides the method of embodiment 21, wherein the construct prevents or minimizes binding of FGF23 to α-Klotho on the surface of the mammal's cell.

Embodiment 23 provides the method of any of embodiments 21-22, wherein the disease or disorder includes hypophosphatemia and/or tumor-induced osteomalacia.

Embodiment 24 provides the method of any of embodiments 21-23, wherein the mammal is human.

Embodiment 25 provides the method of any of embodiments 21-24, wherein the construct is administered by an administration route selected from the group consisting of inhalational, oral, rectal, vaginal, parenteral, intracranial, topical, transdermal, pulmonary, intranasal, buccal, ophthalmic, intrathecal, and intravenous.

Embodiment 26 provides the method of any of embodiments 21-24, wherein the construct of any of embodiments 1-15 or a precursor thereof is delivered on an encoded vector, wherein the vector encodes the construct or precursor thereof and, upon administration of the vector to the subject, the construct is transcribed and translated from the vector.

Embodiment 27 provides the method of any of embodiments 21-26, wherein the mammal is further administered at least one additional drug that treats or prevents the disease and/or disorder.

Embodiment 28 provides the method of embodiment 27, wherein the construct and the at least one additional drug are co-administered.

Embodiment 29 provides the method of any of embodiments 27-28, wherein the construct and the at least one additional drug are co-formulated.

### BRIEF DESCRIPTION OF THE DRAWINGS

For the purpose of illustrating the invention, certain embodiments of the invention are depicted in the drawings. However, the invention is not limited to the precise arrangements and instrumentalities of the embodiments depicted in the drawings.

FIGS. 1A-1D illustrate the finding that the C-terminal tail of FGF23 contains two distinct regions that specifically bind to α-Klotho. FIG. 1A: Schematic representation of FGF19, FGF21, FGF23, and FGF1. Signal peptide (SP) is the first section, the FGFR binding domain is the second section, the β Klotho binding regions in the C-terminal tails of FGF19 and FGF21 are the last sections, and the tandem repeats in the C-terminal tail of FGF23 are labeled R1 and R2, respectively. FIG. 1B: Sequence alignments and comparison of the C-terminal tails of human FGF19 and FGF21 to the first (R1) and second (R2) repeats of human FGF23 C-terminal tail. The DPL motif of FGF19, FGF21, and FGF23, critical for binding D1 of α- and β-Klotho, is the highlighted residues on the left and the SPS sugar mimicking motif in FGF19 and FGF21, critical for binding to the pseudo-substrate binding pocket in D2 of β-Klotho, is the highlighted section on the right. FIG. 1C: Representative BLI sensorgrams illustrating the binding of GST fusion of the C terminal tail of FGF23 (GST-FL), R1 (GST-R1) and R2 (GST-R2) to sKLA. Biosensors coated with anti-GST antibody were used to capture GST-fused FGF23 peptide fragments and dipped into solutions containing a series of concentrations of sKLA (6.25, 12.5, 25, 50, 100, and 200 nM). Sensorgrams were fitted with a 1:1 ligand:receptor binding model to calculate dissociation constants and kinetic parameters. FIG. 1D: Summary of kinetic parameters and dissociation constants of BLI measurements. Data are presented as mean values±S.D. from 3 independent experiments.

FIGS. 2A-2H illustrate the finding that FGF23-WT and FGF23 variants with either R1 or R2 induce similar cellular responses. FIG. 2A: Schematic representation of the C-terminal tails of FGF23 variants used for cell stimulation. R1 and R2 are labeled. Mutations in R1 and/or R2 of the FGF23 C-tail that abolish binding to α-Klotho, are marked. FIGS. 2B-2H: Comparison of FGF23-WT or FGF23 variants induced tyrosine phosphorylation of FRS2α and MAPK response. HEK293 cells stably expressing FGFR1c together with α-Klotho were left unstimulated or stimulated with increasing concentrations of FGF23 or FGF23 variants as indicated for 10 minutes at 37° C. Cell lysates were subjected to SDS-PAGE and analyzed for tyrosine phosphorylation of FRS2α and MAPK activation by immunoblotting with antibodies for pFRS2, pMAPK, respectively and with anti-MAPK as a control.

FIGS. 3A-3H illustrate the finding that similar inhibition of FGF23 induced stimulation of cells takes place when the cells are treated with Fc-R2 or the cells are treated with Fc-FL or Fc-R1; further, cysteine residues flanking R2 in FGF23 C-tail form intramolecular disulfide bridge. FIGS. 3A-3B: Schematic representation (FIG. 3A) and SDS-PAGE analyzes (FIG. 3B) under reducing (R) or non-reducing (NR) conditions of Fc-FL, Fc-R1 or Fc-R2. The Fc moiety, R1 and R2 are labeled. FIGS. 3C-3E: HEK 293 cells stably expressing FGFR1c and α-Klotho were incubated with increasing concentrations (as indicated) of Fc-FGF23 full length tail (Fc-FL), Fc-R1, or Fc-R2 for 45 minutes at 37° C. Cells were then stimulated with FGF23-WT for additional 10 minutes and cell lysates were subjected to SDS-PAGE and analyzed for MAPK stimulation by immunoblotting with anti-pMAPK antibodies. Anti-FGFR1 and anti-MAPK antibodies were used as control for protein loading. FIG. 3F: Schematic representation of C-terminal tails of FGF23-WT and FGF23-CS. R1 and R2 are shown. Cysteine residues (C) and serine residues (S) are highlighted.

FIG. 3G: SDS-PAGE analyses of FGF23-WT and FGF23-CS mutant expressed in E. coli, under reducing (R) and non-reducing conditions (NR). FGF23-WT and FGF23-CS were expressed and purified as described elsewhere herein. While FGF23-CS migrates on SDS-PAGE as single band under both reducing and non-reducing conditions, FGF23-WT migrates as two distinct bands (labeled by the two asterisks) under non-reducing conditions. Both proteins were excised from the gel and subjected to mass-spectrometric analysis. FIG. 3H: SDS-PAGE analyses of FGF23-WT or FGF23-CS mutant expressed in Expi293F cells, under reducing (R) and non-reducing conditions (NR). Unlike E. coli produced FGF23, mammalian produced FGF23 is O-linked glycosylated. Under reducing conditions (R), the upper band (the upper left asterisk) and lower band (the upper left asterisk) present O-linked glycosylated form and a non- or poorly-glycosylated form of FGF23, respectively (see also FIG. 7B). Under non-reducing conditions (NR), both glycosylated (the upper right asterisk) and non-glycosylated FGF23 (the lower right asterisk) migrate faster than the reduced proteins on SDS-PAGE due to the formation of intramolecular disulfide bridge. The migration of FGF23-WT or FGF23-CS (expressed in Expi293F cells) on SDS-PAGE under both reducing and non-reducing conditions is similar and the upper and lower bands represent O-linked glycosylated and non-glycosylated forms of the ligand, respectively.

FIGS. 4A-4F illustrate the finding that FGF23-WT binds simultaneously to two α Klotho molecules, expressed on cell membranes. FIG. 4A: L6 cells stably expressing α-Klotho-FGFR1c chimeric receptors were left unstimulated or stimulated with FGF23-WT or FGF23-R1 expressed in Expi293F cells (left panel), or FGF23-WT and FGF23-R1 expressed in E. coli (right panel), Nb85-Fc fused (left panel) or Fc-R1 (right panel) for 10 minutes at 37° C. Cell lysates of unstimulated or ligands stimulated cells were subjected to SDS-PAGE and analyzed for FRS2α phosphorylation and the activation of MAPK by immunoblotting with anti-pFRS2 and anti-pMAPK antibodies, respectively, and with anti-MAPK as a control.

FIG. 4B: Expanded view of single HaloTag-α-Klotho particles on the surface of living L6 cells imaged by TIRFM. The HaloTag on the extracellular side of α-Klotho was labeled with a cell-impermeant Alexa488 HaloTag ligand. Particle density is 0.21 particles/μm². A single frame (100-ms exposure) at the start of a 10-Hz recording is shown. Scale bar, 5 FIG. 4C: Automated detection and tracking of moving HaloTag-α-Klotho particles during a 10-s recording period. Single-particle tracking was performed as described in materials and methods. Inset, higher magnification. FIG. 4D: Representative single-step photobleaching of HaloTag-α-Klotho particles. Average fluorescence intensity within a 0.55-μm-diameter region surrounding a HaloTag-α-Klotho particle was measured with local background subtraction (using a concentric annular region with inner and outer diameters of 0.55 and 1.1 respectively) and plotted against time. Note similar intensity of the particles before bleaching (highlighted by horizontal lines). FIG. 4E: Representative intensity distributions of HaloTag-α-Klotho in a cell left unstimulated (top panel) or stimulated with FGF23-WT for ˜10 min (bottom panel). Particle densities were 0.26 and 0.05 particles/μm² for unstimulated and stimulated conditions, respectively. Intensities represent the volume under 2D Gaussian fits of the fluorescence of particles. Intensities were taken from the beginning (3 frames) of each recording and their distribution was fitted with a mixed Gaussian model. Black dashed lines, mixed fit. Solid lines, individual components. FIG. 4F: Diffusion coefficient of HaloTag-α-Klotho particles calculated from their mean square displacement in unstimulated cells (18 cells, 5 transfections) and cells stimulated with FGF23-WT (16 cells, 4 transfections), FGF23-R1 (14 cells, 3 transfections), FGF23-R2 (16 cells, 3 transfections) and Nb85-Fc (16 cells, 3 transfections). Error bars indicate mean±SE, ***P<0.0001 by Student's t-test.

FIG. 5A illustrates amino acid sequence alignments of the C-terminal tails of FGF23 from various mammalian species (H. Sapiens (human), amino acid residues 180-251 of SEQ ID NO: 5, M mulatta (rhesus monkey), SEQ ID NO:8, E. caballus (horse), SEQ ID NO:9, L. africana (elephant), SEQ ID NO: 10, B. Taurus (cow), SEQ ID NO: 11, C. lupus familiaris (Dog), SEQ ID NO: 12, M. musculus (mouse), SEQ ID NO: 13, R. norvegicus (rat), SEQ ID NO: 14, M. auratus (hamster), SEQ ID NO: 15, G. gallus (chicken), SEQ ID NO: 16, A. mississippiensis (alligator), SEQ ID NO: 17, X. laevis (frog), SEQ ID NO: 18, D. rerio (zebra fish), SEQ ID NO: 19). Repeat 1 (R1) and repeat 2 (R2) are labeled, and conserved cysteine residues are highlighted. The conserved DPL motif, which is important for Klotho binding, is highlighted, as well. FIG. 5B illustrates amino acid sequence alignments of the C-terminal tails of FGF23 from other vertebrate species. In vertebrates other than mammals only two amino acids (DP) of the DPL motif are conserved and they are highlighted.

FIG. 6 illustrates SDS-PAGE analysis of GST-FL, GST-R1, GST-R2, and GST.

FIG. 7A illustrates MS/MS fragmentation spectrum directly identified the digested fragment of FGF23 expressed in E. coli that contains the Cys206-Cys244 disulfide bond. The corresponding b and y ions mapping to respective fragment ions of the two peptides forming disulfide-linked peptide were highlighted. FIG. 7B illustrates relative amounts of bridged vs non-bridged Cys206 and Cys244 in various FGF23 samples presented by PRM measurement. The unique MS2 ions of high resolution (different traces) were manually inspected with Skyline visualization.

FIG. 8A illustrates MS/MS fragmentation spectrum directly identified the fragment of FGF23 expressed in Expi293F cells (mFGF23-WT) containing the Cys206-Cys244 disulfide bond. The corresponding b and y ions mapping to respective fragment ions of the two peptides forming disulfide-linked peptide were highlighted. FIG. 8B illustrate SDS-PAGE of purified mFGF23-WT, mFGF23-CS (C206S/C244S mutant), and mFGF23-R1 (variant lacking C-terminal residues C206-I251) treated with O-glycosidase and α-(2→3,6,8,9)-neuraminidase. All FGF23 variants were expressed in Expi293 cells.

FIG. 9 illustrates SDS-PAGE analysis of FGF23-WT and FGF23-CS expressed and purified from Expi293 cells, subjected to limited protease digestion with various proteases as indicated.

FIGS. 10A-10D illustrate the finding that FGF23-WT and FGF23-CS bind α-Klotho with similar affinities and activate cell signaling to a similar extent. FIG. 10A: BLI sensorgrams of binding of FGF23-WT, FGF23-CS, FGF23 D188A, and FGF23-CS D188A to sKLA. Biosensors coated with anti-mouse-Fc antibody was used to immobilize anti-Flag antibody followed by capturing Flag-tagged FGF23-WT or FGF23-CS. Biosensors were then dipped into solutions containing a series of concentrations of sKLA (200, 100, 50, 25, 12.5, and 6.25 nM). The resulting sensorgrams were fitted with a 1:1 ligand:receptor binding model to calculate kinetic parameters and dissociation constants. FIGS. 10B-10C: HEK293 cells stably expressing FGFR1c together with α-Klotho were left unstimulated or stimulated with increasing concentrations of FGF23-WT or FGF23-CS mutant, as indicated, for 10 minutes at 37° C. Cell lysates were subjected to SDS-PAGE and analyzed for serine phosphorylation of FGFR1, tyrosine phosphorylation of FRS2 and for MAPK activation by immunoblotting with anti-FGFR1-pS, anti-pFRS2 and anti-pMAPK antibodies, respectively and with anti-MAPK as a control. FIG. 10D: Schematic diagram depicting the bivalency of the C-terminal tail of FGF23 binding to α-Klotho.

FIGS. 11A-11C illustrate non-limiting detection of single molecules of free Alexa488 HaloTag ligand by TIRFM. FIG. 11A: TIRFM image of Alexa488 HaloTag ligand spotted onto glass. Scale bar, 2.5 μm. FIG. 11B: Representative single-step photobleaching of Alexa488 HaloTag ligand particles. Average fluorescence intensity within a 0.55-μm-diameter region surrounding a particle was measured with local background subtraction (using a concentric annulus) and plotted against time. Note similar intensity of the three examples before and after bleaching (horizontal lines). FIG. 11C: Intensity distribution of Alexa488 HaloTag ligand particles. The distribution was best fitted with a single Gaussian (the curve of solid line). For intensity distribution analyses, intensities were calculated by fitting the fluorescence of particles with 2D Gaussian functions and taking the volume under the fit.

FIG. 12 illustrates examples of intensity changes of individual tracks of HaloTag-α-Klotho particles compatible with reversible dimer formation. Arrows highlight the abrupt doubling of particle intensity. The starting intensity likely corresponds to that of a single molecule, based on the intensity distribution of particles (FIG. 4E).

FIG. 13 illustrates a non-limiting example of HaloTag-α-Klotho particles transiently merging. Image sequence of consecutive frames showing two monomers (arrowheads) diffuse towards each other, merge and then dissociate back into monomers. Color lookup table (bottom).

### DETAILED DESCRIPTION OF THE INVENTION

FGF23 is a bone-derived hormone that play as an important physiological regulator of renal Pi excretion. Transgenic mice that overexpresses FGF23 develop hypophosphatemia, whereas FGF23-knockout mice develop hyperphosphatemia, which can be reversed by systemic injection of human FGF23.

Importantly, these in vivo actions of FGF23 require the presence of α-Klotho. Injection of FGF23 into α-Klotho-knockout mice or FGF23/α-Klotho-double knockout mice did not affect the serum phosphate level. Like other endocrine FGFs, FGF23 exhibits isoform specificity for FGFRs—it binds and activates Mc isoform of FGFR1 and FGFR3, as well as FGFR4 which only exhibits a single isoform.

FGF23 is associated with a number of human diseases related to dysregulation of phosphate metabolism. X-linked hypophosphatemia (XLH) is an inherited disorder where PHEX (phosphate regulating gene with homologies to endopeptidases located on the X chromosome) contains loss-of-function mutation, and the consequence of this mutation is the elevation of circulating FGF23. Similarly, increased level of FGF23 was observed in autosomal recessive hypophosphatemic rickets 1 (ARHR1) or autosomal recessive hypophosphatemic rickets 2 (ARHR2) patients that carries mutations in DMP-1 or ENPP-1, respectively. In autosomal dominant hypophosphatemic rickets (ADHR), gain-of-function mutations in FGF23 (such as, but not limited to, R176Q and/or R179Q) prevent natural proteolytic cleavages at these sites to make two inactive fragments of FGF23. Without wishing to be limited by any theory, such cleavage can represent a mechanism of down-regulation. Cancers harboring tumors that produce high levels of FGF23 lead to tumor-induced osteomalacia (TIO), which can be reversed by surgical removal of the tumors secreting high FGF23 levels. While increased activities of FGF23 are observed in patients with disorders mentioned above, reduced activity of FGF23 has been also found in patients of hyperphosphosphatemic familial tumoral calcinosis (HFTC). A homozygous loss-of-function mutation in KLA, H193R, was also found in a HFTC patient.

The present disclosure relates in part to the discovery that the C-terminal tail of FGF23 contains two tandem repeats, each of which binds with high affinity to α-Klotho. This is in contrast with FGF19 and FGF21, whose C-terminal tails contain a single binding site to β-Klotho. Engineered FGF23 variants containing each of the two repeats or both repeats bind specifically to α-Klotho and stimulate cell signaling to a similar extent. Further, the present studies show that two cysteine residues flanking the second C-terminal repeat form a disulfide bridge in FGF23 secreted by mammalian cells. However, both oxidized or reduced forms of FGF23 exhibit similar α-Klotho binding characteristics and similar cellular stimulatory activities. Further, FGF23 WT induces MAPK activation in cells expressing chimeric α-Klotho-FGFR proteins, and TIRFM imaging of individual α-Klotho molecules on the cell surface demonstrates that FGF23 has the capacity for simultaneous binding to two α-Klotho molecules. These insights reveal the complexity of FGF23 regulation and its role in assembling the FGF23/FGFR/α-Klotho signaling complex.

In certain embodiments, the present invention provides a construct comprising the R2 region of FGF23 (amino acids 212-239 of SEQ ID NO:5). In other embodiments, the construct functions as FGF23 antagonist by blocking FGF23 binding to α-Klotho and cell signaling via FGFR activation. In yet other embodiments, the construct prevents FGFR activation. In yet other embodiments, the construct of the present disclosure can be used to treat diseases or disorders related to FGF23 dysregulation and/or overexpression, such as but not limited to phosphate metabolism disorders. The invention further provides method of treating, ameliorating, and/or preventing endocrine FGF-related diseases or disorders in a mammal in need thereof.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, ±5%, ±1%, or ±0.1% from the specified value, as such variations are appropriate to perform the disclosed methods.

As used herein, the term “ALB” or “albumin” refers to a serum albumin protein. In certain embodiments, albumin refers to human serum albumin. Usage of other albumins, such as bovine serum albumin, equine serum album and porcine serum albumin, are also contemplated within the invention.

As used herein, the term “α-Klotho” or “KLA” refers to the protein of amino sequence of SEQ ID NO:1 (UniProtKB: Q9UEF7):

```
                10 20 30 40
MPASAPPRRP RPPPPSLSLL LVLLGLGGRR LRAEPGDGAQ
                50 60 70 80
TWARFSRPPA PEAAGLFQGT FPDGELWAVG SAAYQTEGGW
                90 100 110 120
QQHGKGASIW DTFTHHPLAP PGDSRNASLP LGAPSPLQPA
              130 140 150 160
TGDVASDSYN NVERDTEALR ELGVTHYRES ISWARVLPNG
              170 180 190 200
SAGVPNREGL RYYRRLLERL RELGVQPVVT LYHWDLPQRL
              210 220 230 240
QDAYGGWANR ALADHERDYA ELCFRHEGGQ VKYWITIDNP
              250 260 270 280
YVVAWHGYAT GRLAPGIRGS PRLGYLVAHN LLLAHAKVWH
              290 300 310 320
LYNTSFRPTQ GGQVSIALSS HWINPRRMTD HSIKECQKSL
              330 340 350 360
DEVLGWFAKP VFIDGDYPES MKNNLSSILP DFTESEKKFI
              370 380 390 400
KGTADFFALC FGPTLSFQLL DPHMKFRQLE SPNLRQLLSW
              410 420 430 440
IDLEFNHPQI FIVENGWEVS GTTKRDDAKY MYYLKKFIME
              450 460 470 480
TLKAIKLDGV DVIGYTAWSL MDGFEWHRGY SIRRGLFYVD
              490 500 510 520
FLSQDKMLLP KSSALFYQKL IEKNGFPPLP ENQPLEGTFP
              530 540 550 560
CDFAWGVVDN YIQVDTTLSQ FTDLNVYLWD VHHSKRLIKV
              570 580 590 600
DGVVTKKRKS YCVDFAAIQP QIALLQEMHV THERESLDWA
              610 620 630 640
LILPLGNQSQ VNHTILQYYR CMASELVRVN ITPVVALWQP
              650 660 670 680
MAPNQGLPRL LARQGAWENP YTALAFAEYA RLCFQELGHH
              690 700 710 720
VKLWITMNEP YTRNMTYSAG HNLLKAHALA WHVYNEKFRH
              730 740 750 760
AQNGKISIAL QADWIEPACP FSQKDKEVAE RVLEFDIGWL
              770 780 790 800
AEPIFGSGDY PWVMRDWLNQ RNNELLPYFT EDEKKLIQGT
              810 820 830 840
FDFLALSHYT TILVDSEKED PIKYNDYLEV QEMTDITWLN
              850 860 870 880
SPSQVAVVPW GLRKVLNWLK FKYGDLPMYI ISNGIDDGLH
              890 900 910 920
AEDDQLRVYY MQNYINEALK AHILDGINLC GYFAYSENDR
              930 940 950 960
TAPREGLYRY AADQFEPKAS MKHYRKIIDS NGFPGPETLE
              970 980 990 1000
RFCPEEFTVC TECSFFHTRK SLLAFIAFLE FASIISLSLI
            1010
FYYSKKGRRS YK
```

As used herein, the term “β-Klotho” or “KLB” refers to the protein of amino sequence of SEQ ID NO:2 (UniProtKB: Q86Z14):

```
                10 20 30 40
MKPGCAAGSP GNEWIFFSTD EITTRYRNTM SNGGLQRSVI
                50 60 70 80
LSALILLRAV TGFSGDGRAI WSKNPNFTPV NESQLFLYDT
                90 100 110 120
FPKNFFWGIG TGALQVEGSW KKDGKGPSIW DHFIHTHLKN
              130 140 150 160
VSSINGSSDS YIFLEKDLSA LDFIGVSFYQ FSISWPRLFP
              170 180 190 200
DGIVTVANAK GLQYYSTLLD ALVLRNIEPI VTLYHWDLPL
              210 220 230 240
ALQEKYGGWK NDTIIDIEND YATYCFQMEG DRVKYWITIH
              250 260 270 280
NPYLVAWHGY GTGMHAPGEK GNLAAVYTVG HNLIKAHSKV
              290 300 310 320
WHNYNTHERP HQKGWLSITL GSHWIEPNRS ENTMDIFKCQ
              330 340 350 360
QSMVSVLGWF ANPIHGDGDY PEGMRKKLES VLPIFSEAEK
              370 380 390 400
HEMRGTADFF AFSFGPNNEK PLNTMAKMGQ NVSLNLREAL
              410 420 430 440
NWIKLEYNNP RILIAENGWF TDSRVKTEDT TAIYMMKNFL
              450 460 470 480
SQVLQAIRLD EIRVEGYTAW SLLDGFEWQD AYTIRRGLFY
              490 500 510 520
VDENSKQKER KPKSSAHYYK QIIRENGESL KESTPDVQGQ
              530 540 550 560
FPCDESWGVT ESVLKPESVA SSPQFSDPHL YVWNATGNRL
              570 580 590 600
LHRVEGVRLK TRPAQCTDFV NIKKQLEMLA RMKVTHYRFA
              610 620 630 640
LDWASVLPTG NLSAVNRQAL RYYRCVVSEG LKLGISAMVT
              650 660 670 680
LYYPTHAHLG LPEPLLHADG WLNPSTAEAF QAYAGLCFQE
              690 700 710 720
LGDLVKLWIT INEPNRLSDI YNRSGNDTYG AAHNLLVAHA
              730 740 750 760
LAWRLYDRQF RPSQRGAVSL SLHADWAEPA NPYADSHWRA
              770 780 790 800
AERFLQFEIA WFAEPLEKTG DYPAAMREYI ASKHRRGLSS
              810 820 830 840
SALPRLTEAE RRLLKGTVDF CALNHFTTRF VMHEQLAGSR
              850 860 870 880
YDSDRDIQFL QDITRLSSPT RLAVIPWGVR KLLRWVRRNY
              890 900 910 920
GDMDIYITAS GIDDQALEDD RLRKYYLGKY LQEVLKAYLI
              930 940 950 960
DKVRIKGYYA FKLAEEKSKP RFGFFTSDEK AKSSIQFYNK
              970 980 990 1000
VISSRGFPFE NSSSRCSQTQ ENTECTVCLF LVQKKPLIFL
            1010
GCCFFSTLVL LLSIAIFQRQ KRRKEWKAKN LQHIPLKKGK RVVS
```

By the term “applicator,” as the term is used herein, is meant any device including, but not limited to, a hypodermic syringe, a pipette, and the like, for administering the compounds and compositions of the invention.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule that are matched with an anti-codon region of a transfer RNA molecule during translation of the mRNA molecule or that encode a stop codon. The coding region may thus include nucleotide residues corresponding to amino acid residues that are not present in the mature protein encoded by the mRNA molecule (e.g., amino acid residues in a protein export signal sequence).

A “constitutive” promoter is a nucleotide sequence that, when operably linked with a polynucleotide that encodes or specifies a gene product, causes the gene product to be produced in a cell under most or all physiological conditions of the cell.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound which is sufficient to provide a beneficial effect to the subject to which the compound is administered.

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

As used herein “endogenous” refers to any material from or produced inside an organism, cell, tissue or system. As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression may be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “Fc” refers to a human IgG (immunoglobulin) Fc domain. Subtypes of IgG such as IgG1, IgG2, IgG3, and IgG4 are contemplated for usage as Fc domains.

As used herein, the “Fc region” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “FcRn Receptor” refers to the neonatal Fc receptor (FcRn), also known as the Brambell receptor, which is a protein that in humans is encoded by the FCGRT gene. An FcRn specifically binds the Fc domain of an antibody. FcRn extends the half-life of IgG and serum albumin by reducing lysosomal degradation in endothelial cells. IgG, serum albumin, and other serum proteins are continuously internalized through pinocytosis. Generally, serum proteins are transported from the endosomes to the lysosome, where they are degraded. FcRn-mediated transcytosis of IgG across epithelial cells is possible because FcRn binds IgG at acidic pH (<6.5) but not at neutral or higher pH. IgG and serum albumin are bound by FcRn at the slightly acidic pH (<6.5), and recycled to the cell surface where they are released at the neutral pH (>7.0) of blood. In this way IgG and serum albumin avoid lysosomal degradation.

The Fc portion of an IgG molecule is located in the constant region of the heavy chain, notably in the CH2 domain. The Fc region binds to an Fc receptor (FcRn), which is a surface receptor of a B cell and also proteins of the complement system. The binding of the Fc region of an IgG molecule to an FcRn activates the cell bearing the receptor and thus activates the immune system. The Fc residues critical to the mouse Fc-mouse FcRn and human Fc-human FcRn interactions have been identified (Dall'Acqua et al., 2002, J. Immunol. 169(9):5171-80). An FcRn binding domain comprises the CH2 domain (or a FcRn binding portion thereof) of an IgG molecule.

As used herein, the term “fragment,” as applied to a nucleic acid, refers to a subsequence of a larger nucleic acid. A “fragment” of a nucleic acid can be at least about 5, 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 5, 10, 20, 50, 100, 200, 300 or 400 amino acids in length (and any integer value in between).

As used herein, the term “FGF19” refers to a polypeptide of amino acid sequence of SEQ ID NO:3 (UniProtKB: 095750):

```
                10 20 30 40
MRSGCVVVHV WILAGLWLAV AGRPLAFSDA GPHVHYGWGD
                50 60 70 80
PIRLRHLYTS GPHGLSSCEL RIRADGVVDC ARGQSAHSLL
                90 100 110 120
EIKAVALRTV AIKGVHSVRY LCMGADGKMQ GLLQYSEEDC
              130 140 150 160
AFEEEIRPDG YNVYRSEKHR LPVSLSSAKQ RQLYKNRGEL
              170 180 190 200
PLSHELPMLP MVPEEPEDLR GHLESDMESS PLETDSMDPE
              210
GLVTGLEAVR SPSFEK
```

As used herein, the term “FGF21” refers to a polypeptide of amino acid sequence of SEQ ID NO:4 (UniProtKB: Q9NSA1):

```
                10 20 30 40
MDSDETGFEH SGLWVSVLAG LLLGACQAHP IPDSSPLLQF
                50 60 70 80
GGQVRQRYLY TDDAQQTEAH LEIREDGTVG GAADQSPESL
                90 100 110 120
LQLKALKPGV IQILGVKTSR FLCQRPDGAL YGSLHEDPEA
              130 140 150 160
CSFRELLLED GYNVYQSEAH GLPLHLPGNK SPHRDPAPRG
              170 180 190 200
PARELPLPGL PPALPEPPGI LAPQPPDVGS SDPLSMVGPS
              210
QGRSPSYAS
```

As used herein, the term “FGF23” refers to a polypeptide of amino acid sequence of SEQ ID NO:5 (UniProtKB: Q9GZV9):

```
                10 20 30 40
MLGARLRLWV CALCSVCSMS VLRAYPNASP LLGSSWGGLI
                50 60 70 80
HLYTATARNS YHLQIHKNGH VDGAPHQTIY SALMIRSEDA
                90 100 110 120
GFVVITGVMS RRYLCMDERG NIFGSHYFDP ENCREQHQTL
              130 140 150 160
ENGYDVYHSP QYHELVSLGR AKRAFLPGMN PPPYSQFLSR
              170 180 190 200
RNEIPLIHEN TPIPRRHTRS AEDDSERDPL NVLKPRARMT
              210 220 230 240
PAPASCSQEL PSAEDNSPMA SDPLGVVRGG RVNTHAGGTG
              250
PEGCRPFAKF I
```

In certain embodiments, amino acid residues 1-24 of SEQ ID NO:5 correspond to the signal peptide of FGF23. In certain embodiments, amino acid residues 25-162 of SEQ ID NO:5 correspond to the FGF domain of FGF23. In certain embodiments, amino acid residues 180-205 of SEQ ID NO:5 correspond to the R1 region of FGF23. In certain embodiments, amino acid residues 212-239 of SEQ ID NO:5 correspond to the R2 region of FGF23.

“Gene transfer” and “gene delivery” refer to methods or systems for reliably inserting a particular nucleic acid sequence into targeted cells.

As used herein, the term “in vivo half-life” for a construct contemplated within the disclosure refers to the time required for half the quantity administered in the animal to be cleared from the circulation and/or other tissues in the animal. When a clearance curve of a construct is constructed as a function of time, the curve is usually biphasic with a rapid α-phase (which represents an equilibration of the administered molecules between the intra- and extra-vascular space and which is, in part, determined by the size of molecules), and a longer (which represents the catabolism of the molecules in the intravascular space). In certain embodiments, the term “in vivo half-life” in practice corresponds to the half-life of the molecules in the β-phase.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

As used herein, the term “immunoglobulin” or “Ig” is defined as a class of proteins that function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitor-urinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the co-existing materials of its natural state is “isolated.” An isolated nucleic acid or protein may exist in substantially purified form, or may exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

As used herein, the term “modulate” is meant to refer to any change in biological state, i.e. increasing, decreasing, and the like. For example, the term “modulate” may be construed to refer to the ability to regulate positively or negatively the expression, stability or activity of a target protein, including but not limited to transcription of a target protein mRNA, stability of a target protein mRNA, translation of a target protein mRNA, target protein stability, target protein post-translational modifications, target protein activity, or any combination thereof. Further, the term modulate may be used to refer to an increase, decrease, masking, altering, overriding or restoring of activity, including but not limited to, target protein activity.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of a composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, intracranial and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

“Pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Suitable pharmaceutically acceptable acid addition salts may be prepared from an inorganic acid or from an organic acid.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus. As used herein, a “peptidomimetic” is a compound containing non-peptidic structural elements that is capable of mimicking the biological action of a parent peptide. A peptidomimetic may or may not comprise peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant DNA” as used herein is defined as DNA produced by joining pieces of DNA from different sources. The term “recombinant polypeptide” as used herein is defined as a polypeptide produced by using recombinant DNA methods.

The term “RNA” as used herein is defined as ribonucleic acid.

By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, a “subject” refers to a human or non-human mammal. Non-human mammals include, for example, livestock and pets, such as ovine, bovine, porcine, canine, feline and murine mammals. In certain embodiments, the subject is human.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

As used herein, the term “treatment” or “treating” is defined as the application or administration of a therapeutic agent, i.e., a composition useful within the invention (alone or in combination with another pharmaceutical agent), to a subject, or application or administration of a therapeutic agent to an isolated tissue or cell line from a subject (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder or the potential to develop the disease or disorder. Such treatments may be specifically tailored or modified, based on knowledge obtained from the field of pharmacogenomics. An appropriate therapeutic amount in any individual case may be determined by one of ordinary skill in the art using routine experimentation.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

Abbreviation used herein include: FGF, fibroblast growth factor; FGFR, fibroblast growth factor receptor; HSPG, heparan sulfate proteoglycans; RTK, receptor tyrosine kinase; sKLA, extracellular domain of α-Klotho; sKLB, extracellular domain of β-Klotho.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Disclosure

The prevailing model at the time of the invention was that FGF23 binding promotes the assembly of a signaling complex on the cell membrane composed of FGFR and α-Klotho receptor. The FGF domain of FGF23 binds to the extracellular domain of FGFR and the C-terminal tail binds to the extracellular domain of α-Klotho. Biochemical and structural analyses revealed a similar mechanism of action of FGF19 and FGF21, i.e. the FGF moieties of FGF19 or FGF21 bind to the extracellular domains of FGFRs and their C-terminal regions bind to the extracellular domain of β-Klotho. The prevailing thought was that Klotho proteins function as co-receptors of endocrine FGFs similar to the role played by HSPG in cell signaling by canonical FGFs. However, as the binding affinities of the C-terminal tails of endocrine FGFs towards Klotho receptors is 1000-10,000 fold stronger than the binding affinities of their FGF-moieties towards FGFRs, Klotho proteins can function as the primary surface receptors for endocrine FGFs, whereas FGFRs function as a catalytic subunit of the assembled activated signaling complex. In certain non-limiting embodiments, HSPG molecules on the cell membrane can facilitate conversion of a ternary FGF23/α-Klotho/FGFR complex into to an FGF23/α-Klotho/FGFR hexamer with stimulated tyrosine kinase activity.

As demonstrated herein, the C-terminal tail of FGF23, which is a region responsible for α-Klotho binding, contains two tandem repeats, R1 and R2, that function as two distinct ligands for α-Klotho. FGF23 variants with a single α-Klotho binding site, FGF23-R1, FGF23-R2 or FGF23-WT with both R1 and R2, bind to α-Klotho with similar binding affinity and stimulate tyrosine phosphorylation and MAPK response. R2 is flanked by two cysteines that form a disulfide bridge in FGF23-WT; disulfide bridge formation in FGF23-WT is dispensable for α-Klotho binding and for cell signaling via FGFRs. Further, FGF23-WT stimulates dimerization and activation of a chimeric receptor molecule composed of the extracellular domain of α-Klotho fused to the cytoplasmic domain of FGFR and employ total internal reflection fluorescence (TIRF) microscopy to visualize individual α-Klotho molecules on the cell surface. These experiments demonstrate that FGF23-WT can act as a bivalent ligand of α-Klotho in cell membrane. In certain embodiments, an engineered R2-containing construct (such as but not limited to Fc-R2) acts as an FGF23-antagonist offering new pharmacological intervention for treating diseases caused by excessive FGF23 abundance or activity.

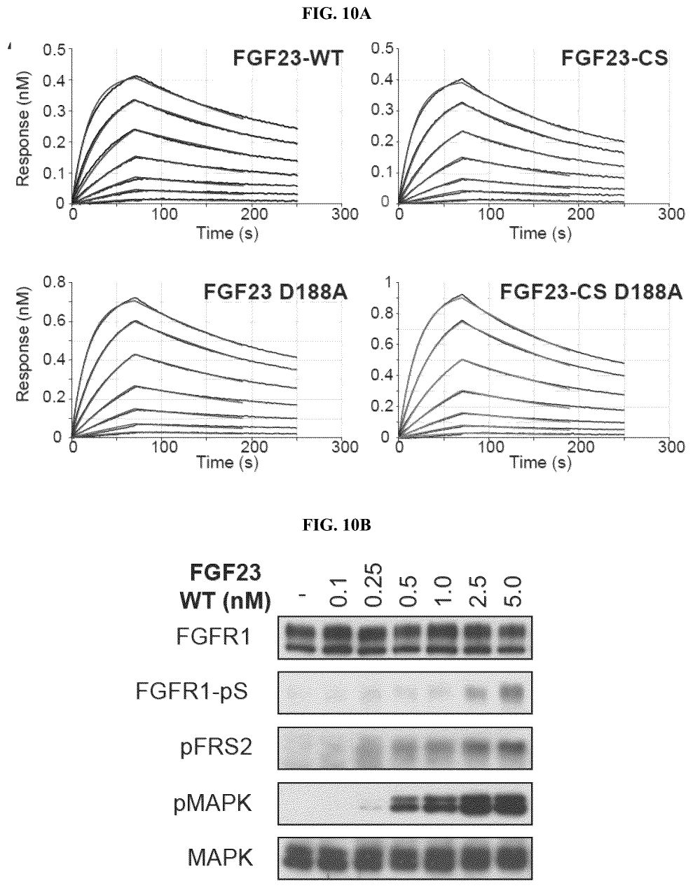

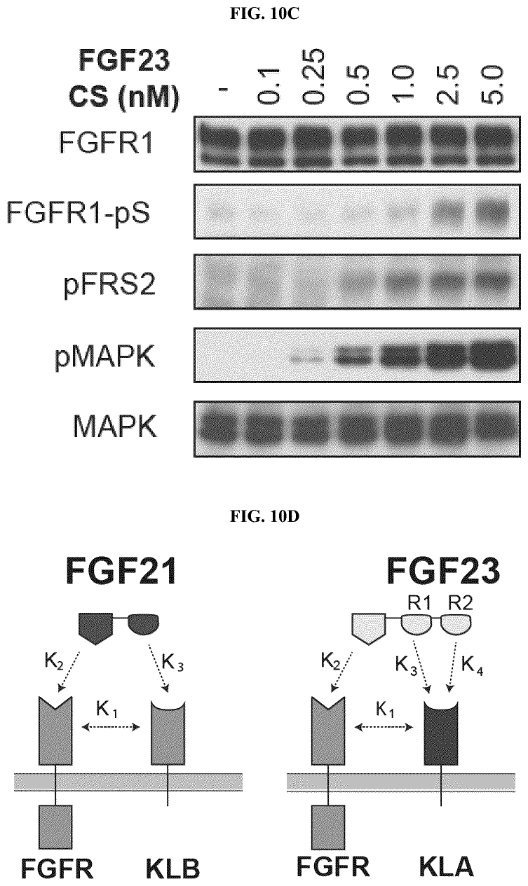

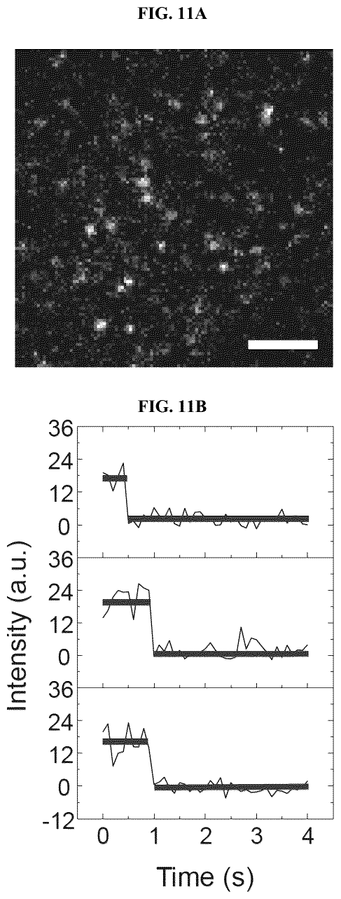

The schematic diagram presented in FIG. 10D depicts interactions between endocrine FGF molecules, FGFRs, and Klotho proteins. Three separate binding events were identified. Dissociation constant K1 for hetero-dimerization of FGFR1c with β-Klotho, dissociation constant K2 for binding of the FGF moiety of FGF21 to FGFR1c, and a dissociation constant K3 for binding of the C-terminal tail of FGF21 to β-Klotho. Binding measurements of each separate association revealed that K1 is ˜1 μM, K2 is ˜100 μM, and K3 in the ˜20 nM range. The present studies demonstrated that the C-terminal tail of FGF23 contains, in addition to the previously identified α-Klotho binding site (R1), a second distinct binding site towards α-Klotho designated R2. Engineered FGF23 containing a single α-Klotho binding site, FGF23-R1 or FGF23-R2 as well as FGF23-WT (with both R1 and R2) bind to sKLA with similar dissociation constants and stimulate similar tyrosine phosphorylation of FRS2α and MAPK response in cells expressing FGFR1c together with α-Klotho. The present studies further demonstrated that an FGF23 variant with an inactive R1 in the context of full-length C-terminal tail utilizes R2 for α-Klotho binding and stimulation of cell signaling by FGFR activation.

In one aspect, FGF23-WT can act as a bivalent ligand of α-Klotho, in certain embodiments based on dimerization and activation of a chimeric receptor composed of the extracellular domain of α-Klotho fused to the cytoplasmic domain of FGFR1 and visualization of individual α-Klotho molecules on the cell surface using TIRF microscopy before and after FGF23 stimulation. The schematic diagram presented in FIG. 10D depicts interactions taking place between FGF23, FGFR1c, and α-Klotho. A difference between the interactions mediated by FGF23 to the interactions mediated by FGF21 (or FGF19) is that the C-terminal tail of FGF23 contains tandem repeats designated R1 and R2 which function as distinct, high affinity ligands for α-Klotho. Yet, since FGF23-R1, FGF23-R2 and FGF23-WT bind to sKLA with similar dissociation constants and are capable of inducing similar FGFR1c activation and cell signaling, in certain non-limiting embodiments a single R1 or R2 ligand is sufficient for α-Klotho binding and cell stimulation. Moreover, even the bivalent FGF23-WT utilizes a single R1 or R2 for α-Klotho binding and for cell activation. Furthermore, treating mice with either FGF23-WT or with a truncated FGF23-R1-like variant resulted in similar regulation of serum phosphate concentration, demonstrating that an FGF23 molecule with a single α-Klotho binding site is capable of stimulating an in vivo physiological response. Without wishing to be limited by any theory, the bivalency of FGF23 towards α-Klotho may facilitate an efficient assembly of signaling complexes on the cell membrane composed of α-Klotho and FGFRs. With a dissociation constant of K1 of ˜1 μM for α-Klotho binding to FGFR, a bivalent FGF23 molecule may stimulate dimerization between a population of preexisting α-Klotho/FGFR heterodimers with either a free α-Klotho molecule or with another pair of preexisting α-Klotho/FGFR heterodimers. While the binding affinities of R1 and R2 to free α-Klotho are very similar to each other, it is possible that FGF23-WT binding to FGFR1c/α-Klotho may be more constrained and limited to interactions with R1 and that the preference of the R2 ligand is to bring together a free α-Klotho molecule to the signaling complex. Accordingly, the architecture of an FGFR1c/α-Klotho heterodimer may preferentially permit interactions with first repeat R1 while the second repeat R2 flanked by two cysteines connected by disulfide bridge may bind to free α-Klotho molecules or vice-versa.

In certain embodiments, the present invention provides a construct comprising the R2 region of FGF23 (amino acids 212-239 of SEQ ID NO:5). In other embodiments, the construct functions as FGF23-antagonist by blocking α-Klotho binding and cell signaling via FGFR activation. In yet other embodiments, the construct of the present disclosure can be used to treat diseases or disorders related to FGF23 dysregulation and/or overexpression, such as but not limited to phosphate metabolism disorders.

### Compounds and Compositions

In certain embodiments, the present invention provides a construct comprising a polypeptide corresponding to R2 region of FGF23, or a biologically active fragment thereof. The R2 region of FGF23 corresponds to amino acids 212-239 of SEQ ID NO:5, or SAEDN SPMAS DPLGV VRGGR VNTHA GGT. In certain embodiments, the construct comprises an amino acid sequence that is at least about 90% (e.g., at least about 91%, 92%, 93%, 94%, 95%, 96%, 97%, 98%, or 99%) identical to amino acids 212-239 of SEQ ID NO:5.

In certain embodiments, the construct is soluble. In certain embodiments, the construct is recombinant. The polypeptide can be fused with another molecule, such as but not limited to a (poly)polypeptide. In certain embodiments, the construct further comprises a stability enhancing domain, or a biologically active fragment thereof, which is fused to the polypeptide. In certain embodiments, the presence of the stability enhancing domain, or a biologically active fragment thereof, improves half-life, improves solubility, reduces immunogenicity, and/or increases the activity of the polypeptide. In certain embodiments, the stability enhancing domain comprises at least one of albumin, thioredoxin, glutathione S-transferase, and/or a Fc region of an antibody. In certain embodiments, the Fc region is IgG Fc. In certain embodiments, the Fc region is the Fc domain of human immunoglobulin 1 (IgG1), human immunoglobulin 2 (IgG2), human immunoglobulin 3 (IgG3), and/or human immunoglobulin 4 (IgG4).

SEQ ID NO: 6: human IgG1 (Fc)
DKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK
SEQ ID NO: 7: human albumin
MKWVTFLLLLFVSGSAFSRGVFRREAHKSEIAHRYNDLGEQHFKGLVLI
AFSQYLQKCSYDEHAKLVQEVTDFAKTCVADESAANCDKSLHTLFGDKL
CAIPNLRENYGELADCCTKQEPERNECFLQHKDDNPSLPPFERPEAEAM
CTSFKENPTTEMGHYLHEVARRHPYFYAPELLYYAEQYNEILTQCCAEA
DKESCLTPKLDGVKEKALVSSVRQRMKCSSMQKFGERAFKAWAVARLSQ
TFPNADFAEITKLATDLTKVNKECCHGDLLECADDRAELAKYMCENQAT
ISSKLQTCCDKPLLKKAHCLSEVEHDTMPADLPAIAADFVEDQEVCKNY
AEAKDVFLGTFLYEYSRRHPDYSVSLLLRLAKKYEATLEKCCAEANPPA
CYGTVLAEFQPLVEEPKNLVKTNCDLYEKLGEYGFQNAILVRYTQKAPQ
VSTPTLVEAARNLGRVGTKCCTLPEDQRLPCVEDYLSAILNRVCLLHEK
TPVSEHVTKCCSGSLVERRPCFSALTVDETYVPKEFKAETFTFHSDICT
LPEKEKQIKKQTALAELVKHKPKATAEQLKTVMDDFAQFLDTCCKAADK
DTCFSTEGPNLVTRCKDALARSWSHPQFEK

In certain embodiments, the stability enhancing domain is fused with the N-terminus of the polypeptide. In certain embodiments, the stability enhancing domain is fused with the C-terminus of the polypeptide.

In certain embodiments, the stability enhancing domain is directly fused (i.e., without a linker) to the polypeptide. In certain embodiments, the stability enhancing domain is fused through a linker to the polypeptide. In certain embodiments, the linker comprises about 1-18 amino acids and/or 1-20 ethylene glycol and/or propylene glycol units. In certain embodiments, the polypeptide and the stability enhancing domain are linked through a linker comprising about 1-18 amino acids, 1-17 amino acids, 1-16 amino acids, 1-15 amino acids, 1-14 amino acids, 1-13 amino acids, 1-12 amino acids, 1-11 amino acids, 1-10 amino acids, 1-9 amino acids, 1-8 amino acids, 1-7 amino acids, 1-6 amino acids, 1-5 amino acids, 1-4 amino acids, 1-3 amino acids, 1-2 amino acids, or a single amino acid.

In certain embodiments, the C-terminus of the linker fused to the N-terminus of the polypeptide is not one of the following: APASCSQELP (SEQ ID NO:20), PASCSQELP (SEQ ID NO:21), ASCSQELP (SEQ ID NO:22), SCSQELP (SEQ ID NO:23), CSQELP (SEQ ID NO:24), SQELP (SEQ ID NO:25), QELP (SEQ ID NO:26), ELP, LP, P.

In certain embodiments, the N-terminus of the linker fused to the C-terminus of the polypeptide is not one of the following: GPEGCRPFAKF (SEQ ID NO:27), GPEGCRPFAK (SEQ ID NO:28), GPEGCRPFA (SEQ ID NO:29), GPEGCRPF (SEQ ID NO:30), GPEGCRP (SEQ ID NO:31), GPEGCR (SEQ ID NO:32), GPEGC (SEQ ID NO:33), GPEG (SEQ ID NO:34), GPE, GP, G.

In certain embodiments, the construct is further pegylated (fused with a poly(ethylene glycol) chain). In certain embodiments, the construct is further at least partially methylated. In certain embodiments, the construct is further C-terminus amidated.

Also provided herein are nucleic acids that encode any one of the constructs of the disclosure. The disclosure further provides vectors, such as expression vectors, that comprise such nucleic acids. Also provided are a cell, cells, or a plurality of cells (e.g., mammalian cells) that comprise any one of the nucleic acids, vectors, or expression vectors described herein. Also provided are methods for producing a construct of the disclosure, the methods in certain embodiments comprising culturing the cell, cells, or plurality of cells under conditions suitable for expression of the construct by the cell or cells from the nucleic acid, vector, or expression vector. The methods can also include purifying the construct from the cell, cells, or plurality of cells, or from the media in which the cell, cells, or plurality of cells were cultured. In addition, the disclosure provides construct purified by any such methods.

The disclosure further provides an autonomously replicating or an integrative mammalian cell vector comprising a recombinant nucleic acid encoding a construct of the disclosure. In certain embodiments, the vector comprises a plasmid or a virus. In other embodiments, the vector comprises a mammalian cell expression vector. In yet other embodiments, the vector further comprises at least one nucleic acid sequence that directs and/or controls expression of the construct. In yet other embodiments, the recombinant nucleic acid encodes a polypeptide comprising a construct of the disclosure and a signal peptide, wherein the polypeptide is proteolytically processed upon secretion from a cell to yield the construct of the disclosure.

In yet another aspect, the disclosure provides an isolated host cell comprising a vector of the disclosure. In certain embodiments, the cell is a non-human cell. In other embodiments, the cell is mammalian. In yet other embodiments, the vector of the disclosure comprises a recombinant nucleic acid encoding a polypeptide comprising a construct of the disclosure and a signal peptide. In yet other embodiments, the polypeptide is proteolytically processed upon secretion from a cell to yield the construct of the disclosure.

### Gene Therapy

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein.

AAV-Mediated Gene Therapy:

AAV, a parvovirus belonging to the genus Dependovirus, has several features that make it particularly well suited for gene therapy applications. For example, AAV can infect a wide range of host cells, including non-dividing cells. Furthermore, AAV can infect cells from a variety of species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, which is a linear, single-stranded DNA molecule containing approximately 4,700 nucleotides (the AAV-2 genome consists of 4,681 nucleotides, the AAV-4 genome 4,767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells. Because the patient's cells lack the rep and cap genes, as well as the accessory function genes, the rAAV cannot further replicate and package their genomes. Moreover, without a source of rep and cap genes, wtAAV cannot be formed in the patient's cells.

There are eleven known AAV serotypes, AAV-1 through AAV-11 (Mori, et al., 2004, Virology 330(2):375-83). AAV-2 is the most prevalent serotype in human populations; one study estimated that at least 80% of the general population has been infected with wt AAV-2 (Berns and Linden, 1995, Bioessays 17:237-245). AAV-3 and AAV-5 are also prevalent in human populations, with infection rates of up to 60% (Georg-Fries, et al., 1984, Virology 134:64-71). AAV-1 and AAV-4 are simian isolates, although both serotypes can transduce human cells (Chiorini, et al., 1997, J Virol 71:6823-6833; Chou, et al., 2000, Mol Ther 2:619-623). Of the six known serotypes, AAV-2 is the best characterized. For instance, AAV-2 has been used in a broad array of in vivo transduction experiments, and has been shown to transduce many different tissue types including: mouse (U.S. Pat. Nos. 5,858,351; 6,093,392), dog muscle; mouse liver (Couto, et al., 1999, Proc. Natl. Acad. Sci. USA 96:12725-12730; Couto, et al., 1997, J. Virol. 73:5438-5447; Nakai, et al., 1999, J. Virol. 73:5438-5447; and, Snyder, et al., 1997, Nat. Genet. 16:270-276); mouse heart (Su, et al., 2000, Proc. Natl. Acad. Sci. USA 97:13801-13806); rabbit lung (Flotte, et al., 1993, Proc. Natl. Acad. Sci. USA 90:10613-10617); and rodent photoreceptors (Flannery et al., 1997, Proc. Natl. Acad. Sci. USA 94:6916-6921).

The broad tissue tropism of AAV-2 may be exploited to deliver tissue-specific transgenes. For example, AAV-2 vectors have been used to deliver the following genes: the cystic fibrosis transmembrane conductance regulator gene to rabbit lungs (Flotte, et al., 1993, Proc. Natl. Acad. Sci. USA 90:10613-10617); Factor NIII gene (Burton, et al., 1999, Proc. Natl. Acad. Sci. USA 96:12725-12730) and Factor IX gene (Nakai, et al., 1999, J. Virol. 73:5438-5447; Snyder, et al., 1997, Nat. Genet. 16:270-276; U.S. Pat. No. 6,093,392) to mouse liver, dog, and mouse muscle (U.S. Pat. No. 6,093,392); erythropoietin gene to mouse muscle (U.S. Pat. No. 5,858,351); vascular endothelial growth factor (VEGF) gene to mouse heart (Su, et al., 2000, Proc. Natl. Acad. Sci. USA 97:13801-13806); and aromatic 1-amino acid decarboxylase gene to monkey neurons. Expression of certain rAAV-delivered transgenes has therapeutic effect in laboratory animals; for example, expression of Factor IX was reported to have restored phenotypic normalcy in dog models of hemophilia B (U.S. Pat. No. 6,093,392). Moreover, expression of rAAV-delivered NEGF to mouse myocardium resulted in neovascular formation (Su, et al., 2000, Proc. Natl. Acad. Sci. USA 97:13801-13806), and expression of rAAV-delivered AADC to the brains of parkinsonian monkeys resulted in the restoration of dopaminergic function.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include, but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal α-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like.

Also included in the disclosure is a method of treating a mammal having a disease or disorder in an amount effective to provide a therapeutic effect. The method comprises administering to the mammal an rAAV vector encoding the polypeptide of interest. Preferably, the mammal is a human.

Typically, the number of viral vector genomes/mammal which are administered in a single injection ranges from about 1×10⁸ to about 5×10¹⁶. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹¹ to about 5×10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal.

### Methods

In one aspect, the invention includes a method of treating or preventing a disease or disorder in a subject in need thereof.

In certain embodiments, the construct functions as FGF23 antagonist by blocking α-Klotho binding and cell signaling via FGFR activation. In yet other embodiments, the construct prevents FGFR activation. In yet other embodiments, the construct can be used to treat diseases or disorders related to FGF23 dysregulation and/or overexpression, such as but not limited to phosphate metabolism disorders. The invention further provides method of treating, ameliorating, and/or preventing endocrine FGF-related diseases or disorders in a mammal in need thereof.

In certain embodiments, the method comprises administering to the subject a therapeutically effective amount of a construct of the disclosure. Non-limiting examples of diseases or disorders treated or prevented by the method includes various types of hypophosphatemia, such as, but not limited to, X-linked hypophosphatemia (XLH), autosomal recessive hypophosphatemic rickets 1 (ARHR1), hypophosphatemic rickets 2 (ARHR2), and autosomal dominant hypophatemic rickets (ADHR). Further non-limiting examples of diseases or disorders treated or prevented by the method includes tumor-induced osteomalacia (TIO). As the level of FGF23 is highly increased in patients suffering from Chronic Kidney Disease (CKD), inhibitors of α-Klotho or FGF23 can also be used for treatment of CKD patients.

In certain embodiments, the disease or disorder includes hypophosphatemia and/or tumor-induced osteomalacia.

In certain embodiments, the subject is a mammal. In other embodiments, the mammal is human. In yet other embodiments, the construct is administered by an administration route selected from the group consisting of inhalational, oral, rectal, vaginal, parenteral, intracranial, topical, transdermal, pulmonary, intranasal, buccal, ophthalmic, intrathecal, and intravenous. In certain embodiments, the construct or its precursor is delivered on an encoded vector, wherein the vector encodes the construct or its precursor and it is transcribed and translated from the vector upon administration of the vector to the subject.

In certain embodiments, the construct is formulated for administration by an administration route selected from the group consisting of inhalational, oral, rectal, vaginal, parenteral, intracranial, topical, transdermal, pulmonary, intranasal, buccal, ophthalmic, intrathecal, and intravenous.

In certain embodiments, the subject is further administered at least one additional drug that treats the disease and/or disorder. In other embodiments, the construct and the at least one additional drug are co-administered. In yet other embodiments, the construct and the at least one additional drug are co-formulated.

It will be appreciated by one of skill in the art, when armed with the present disclosure including the methods detailed herein, that the invention is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present invention may provide benefit.

### Combination Therapies

The compounds and compositions identified using the methods described here are useful in the methods of the invention in combination with one or more additional compounds useful for treating the diseases or disorders contemplated herein. These additional compounds may comprise compounds identified herein or compounds, e.g., commercially available compounds, known to treat, prevent, or reduce the symptoms of the diseases or disorders contemplated herein.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Pharmaceutical Compositions and Formulations

The invention also encompasses the use of pharmaceutical compositions of the invention to practice the methods of the invention.

Such pharmaceutical compositions may be provided in a form suitable for administration to a subject, and may comprise one or more pharmaceutically acceptable carriers, one or more additional ingredients, or some combination of these. The compositions of the invention may comprise a physiologically acceptable salt, such as a compound contemplated within the invention in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, intracranial, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. The therapeutic formulations may be administered to the patient either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or condition in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition. For example, In certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, intracranial, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction and assaying conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials & Methods:

Unless otherwise noted, all starting materials were obtained from commercial suppliers and used without purification.

### Plasmid Construction

A cDNA encoding for full-length human α-Klotho with a C-terminal HA-tag were amplified by PCR and subcloned into the lentiviral transfer plasmids, pLenti CMV Hygro DEST. In order to generate GST fusion proteins DNA fragments of the C-terminal tail of human FGF23, FL (aa 180-251), R1 (aa 180-205) and R2 (aa 212-243) were amplified by PCR and cloned into pGEX4T1 vector (GE Healthcare). DNA fragments encoding full length human FGF23 (FGF23-WT; aa 25-251), as well as FGF23-R1 (aa 25-205) and FGF23-R2 (aa 25-179 fused to aa 212-243) were amplified by PCR and cloned into the bacterial expression plasmids pET-28a. To reduce proteolytic cleavage arginine 179 was substituted by glutamine (R179Q) in all plasmids. The mammalian expression vector of FGF23 composed of a cDNA encoding the signal peptide of human FGF23 followed by a FLAG tag (DYKDDDDK) and human FGF23 (aa 25-251) was subcloned into modified pOptiVec vector (pCMV). Expression vectors of FGF23 variants harboring point mutations or deletions within the C-tail region were generated following standard site-directed mutagenesis protocol (Quick change). All FGF23 encoding plasmids harbor four mutations (R140A/R143A/R176Q/R179Q) to increase ligand stability. Expression vectors encoding Fc fusion proteins were generated using cDNA fragment encoding the signal peptide of mouse IgG1 (aa 1-20) and human IgG1 (aa 223-449) connected to the C-terminal tail of FGF23, FL (aa 180-251), R1 (aa 180-205) or R2 (aa 212-243) cloned into pCMV. Expression vectors for chimeric receptor composed of the extracellular region of human α-Klotho (aa 1-981) fused to the transmembrane and intracellular regions of human FGFR1c (aa 377-822) were cloned into pBabe-Puro system. Expression vector encoding the entire extracellular domain of human α-Klotho (aa 1-980) fused to a 6xhistidine tag (sKLA) was cloned into the mammalian expression vector pCEP4 (Thermo Fisher Scientific).

### Protein Expression and Purification

Purification of sKLA:

Soluble extracellular domain of α-Klotho (sKLA) (aa 1-980) fused to a 6×His tag was expressed in HEK293 EBNA cells and purified from the cell culture medium. For protein purification cells were maintained in Pro293 serum free medium for 6 days and the harvested medium was centrifugated at 300×g, filtered through 0.45 μM membrane, and incubated with Ni Sepharose Excel resin (GE Healthcare) for 1 hour at 4° C. The resin was then washed with mM HEPES (pH 7.5) containing 150 mM NaCl and 10 mM imidazole. sKLA was eluted from the resin with the same buffer containing 300 mM imidazole. The eluted protein was diluted with 20-fold of 25 mM Tris pH 8.0 and subjected to anion exchange chromatography (MonoQ 5/50 GL, GE Healthcare) with a linear NaCl gradient (0-0.4 M). Fractions containing sKLA were concentrated and applied to a Superose 6 column (GE Healthcare) equilibrated with 25 mM HEPES (pH 7.5) containing 150 mM NaCl.

### Purification of GST-Fusion Proteins:

Plasmids encoding for GST-fusion proteins (GST-FL, GST-R1 and GST-R2) were expressed in BL21-Gold (DE3) competent cells (Agilent) and protein purification was conducted as previously described (Olsen, et al., 2004, Proc Natl Acad Sci USA. 101:935-940).

Purification of FGF23 Variants Expressed in E. coli:

Plasmids expressing the various FGF23 variants were expressed in E. coli BL21 DE3 cells. The ligands were purified from inclusion bodies followed by refolding as previously described (Lin, al., 2007, J. Biol. Chem. 282:27277-27284). Refolded FGF23 proteins were captured on heparin affinity HiTrap column (GE Healthcare), eluted using a linear NaCl gradient (0-2.0 M) and subjected to size-exclusion chromatography using HiLoad 26/600 Superdex 200 (GE Healthcare) with buffer containing 25 mM HEPES and 150 mM NaCl at pH 7.5.

### Purification of FLAG-Tagged FGF23:

For the expression of FLAG-tagged FGF23 or its variants, plasmids were transfected into Expi293F cells (Thermo Fisher Scientific) and were cultured in 125 ml flasks in Expi293F expression medium according to manufacturer's protocol. Cells were maintained in expression medium for 6 days, and the medium was collected and incubated with anti-FLAG M2 agarose affinity gel (Millipore Sigma) for 1 hour at 4° C. The gel was washed with 20 column volumes of 25 mM HEPES (pH 7.5) containing 150 mM NaCl and the ligands were eluted with 100 mM Glycine pH 3.0. The eluted fractions were immediately mixed with 1/10 volume of 1 M Tris-HCl (pH 8.0). Fractions containing FGF23 were concentrated and applied to a Superdex S200 Increase column (GE Healthcare) equilibrated with 25 mM HEPES buffer (pH7.5) containing 150 mM NaCl as a final purification step.

### Purification of Fc-FGF23 C-Tail Fragments:

C-terminal tail fragments of FGF23 fused to IgG1 Fc were expressed in Expi293F cells (using the same protocol described above for the expression of FLAG-tagged FGF23). Proteins were purified using Protein A-Sepharose (Thermo Fisher Scientific) followed by size exclusion chromatography using Superdex S200.

### Cell Growth Medium:

HEK293 cells stably co-expressing wild-type FGFR1c and α-Klotho, were grown in DMEM supplemented with 10% FBS, 100 U/ml Penicillin-Streptomycin, 0.1 mg/ml hygromycin and 1 μg/ml puromycin.

HEK293 EBNA cells expressing sKLA were grown in DMEM medium containing 10% Fetal Bovine Serum (FBS), 100 U/mL Penicillin-Streptomycin, 250 μg/mL G-418 and 200 μg/mL of Hygromycin B.

After cell density reached to 70-80% confluency, the medium was changed to Pro293a-CDM (Lonza) supplemented with 100 U/mL Penicillin-Streptomycin.

Expi293F cells were grown in Expi293 expression medium (Thermo Fisher Scientific). These cells were used for transient expression of the Fc-fusion FGF23 C-tail proteins and the FLAG-FGF23 molecules. L6 cell stably expressing α-Klotho-FGFR1c chimeric receptors were grown in DMEM supplemented with 10% FBS, 100 U/mL Penicillin-Streptomycin and 0.5 μg/ml puromycin.

### Bio-Layer Interferometry (BLI) Measurements

Kinetic parameters and dissociation constants of sKLA binding to the various forms of full length FGF23 or GST-fused C-terminal fragments of FGF23 were studied using Bio-Layer Interferometry (BLI). Octet RED96 system (Pall ForteBio) equipped with anti-mouse IgG Fc (AMC) biosensors was used to study interactions between α-Klotho and FLAG-tagged FGF23. Biosensor tips were loaded with anti-FLAG M2 antibody (Millipore Sigma) at 5 μg/ml for 2 min, washed in BLI buffer (25 mM HEPES, 150 mM NaCl, pH 7.5, 0.002% Tween-20, 1 mg/mL BSA) for 60 s, and then loaded with FLAG-tagged FGF23 at 5 μg/ml for 4 min. Alternatively, anti-GST biosensor tips were loaded with 5 ug/ml GST fused with various FGF23 C-terminal fragments for 15 s. Subsequently, ligand-loaded sensor tips were dipped into microplate wells containing different sKLA concentrations, ranging from 6.25 nM to 200 nM in 2-fold dilutions of BLI buffer. After each binding cycle the sensor tips were regenerated with 10 mM glycine (pH 1.5). The collected data were referenced using a parallel buffer control subtraction, and sensograms were fitted globally to a 1:1 Langmuir binding model using ForteBio Data Analysis 10.0 software provided by the manufacturer.

### Deglycosylation of FGF23 Expressed in Mammalian Cells

Purified FGF23 variants were treated with O-glycosidase and α-(2→3,6,8,9)-neuraminidase (New England BioLabs) for 4 h at 37° C. as directed by manufacturer's protocol.

### Shotgun Proteomic Identification of Disulfide Bridge

Disulfide linked peptide mapping was performed by following a published method (Lu, et al., 2015, Nature Methods 12:329-331) and the pLink software was used for the identification of these peptides (Chen, et al., 2019, Nature Communications 10:3404). Briefly, gel bands were processed by following a standard gel-based digestion protocol (Shevchenko, et al., 2006, Nature Protocols 1:2856-2860) with the modifications of digestion condition, in which pH 6.5 with 10 mM N-ethylmaleimide (NEM) was used to avoid disulfide scrambling (Lu, et al., 2015, Nature Methods 12:329-331). Trypsin (Promega) digestion was performed at 10 ng/μL concentration for the gel bands overnight and Glu-C (New England Biolab) at 5 ng/uL for 8 hours. Approximately 0.5 μs peptide digest was used for each LC-MS measurement, using the shotgun mode on the Orbitrap Fusion Lumos Tribrid mass spectrometer (Thermo Fisher Scientific) instrument that was described previously (Li, et al., 2019, J Am Soc Mass Spectrom 10.1007/s13361-019-02243-1). For pLink (Chen, et al., 2019, Nature Communications 10:3404) identification, the combination of GluC and trypsin was specified and up to 3 miss cleavages were allowed, with all the other setting kept as default. The MS/MS spectrum was annotated by pLabel (Lu, et al., 2018, Biophys Rep 4:68-81).

### Parallel Reaction Monitoring (PRM) Quantification of Disulfide Bridge

Peptide samples of E. coli produced FGF23 were injected to monitor the MS quantitative response by PRM mode for relative quantification for the standard non-miss cleavage peptide containing Cys206-Cys244 (disulfide linked MTAPAPSCQE and GCRPFAK). The theoretical MS1 and MS2 m/z values for the linked peptide were generated by Skyline (MacLean, et al., 2010, Bioinformatics 26:966-968) and imported into the PRM method. The isolation window was set to be 1.4 m/z. The Orbitrap resolution for PRM was set at 30,000, AGC target 1.0e5, maximum injection time 150 ms. A stepped HCD Collison energy of 2% (centered at 28%) was used. The resultant PRM data was imported (MacLean, et al., 2010, Bioinformatics 26:966-968) to Skyline for manual inspection.

### Limited Proteolysis

Limited proteolysis of FGF23-WT and FGF23-CS that were produced in mammalian cells were performed using Proti-Ace Kit (Hampton Research) under manufacture recommendations. Digested samples were analyzed by SDS-PAGE followed by Coomassie blue staining.

### Total Internal Reflection Fluorescence Microscopy

For single-molecule imaging experiments, L6 cells were plated on 35-mm glass-bottom dishes (MatTek Corporation) at a density of 2.5×10 5 cells per dish and transfected with 0.25 μg HaloTag-α-Klotho plasmid the next day using Lipofectamine 3000 reagent (Invitrogen), according to the manufacturer's instructions. Cells were labeled with 0.25 μM Alexa488 HaloTag ligand (Promega) for 15 min at 37° C. and then washed three times with phenol-red-free DMEM medium (imaging media). After labeling, cells were immediately imaged at 37° C. and 5% CO₂ in a cage incubator (OkoLab) housing a Nikon Eclipse Ti2 microscope (Nikon) equipped with a motorized Ti-LA-HTIRF module with a 15-mW LU-N4 488 laser, using a CFI Plan Apochromat Lambda 100×/1.45 Oil TIRF objective and a Prime95B cMOS camera (110-nm pixel size; Teledyne Photometrics). Images were acquired using a 100-ms exposure time at 10 Hz with the laser power set at 100%. The penetration depth of the evanescent field was ˜118 nm.

### Automated Single-Particle Tracking

Particles were localized and tracked using the Matlab software GaussStorm. Briefly, particles were automatically detected by application of a bandpass filter to remove noise, followed by convolution with a Gaussian kernel, and then the selection of above-threshold pixels. Particles were then fitted with elliptical two-dimensional Gaussian functions, which yielded their intensities expressed as the volume under the curve, as well as their positions with subpixel accuracy. Particles were tracked frame to frame using a tracking algorithm with a tracking window of 8 pixels between consecutive frames. The distribution of the displacements of single particles was used to calculate mean diffusion coefficient in a field of view encompassing an entire cell.

### Example 1: The C-Terminal Tail of Mammalian FGF23 Contains Two Separate α-Klotho Binding Regions

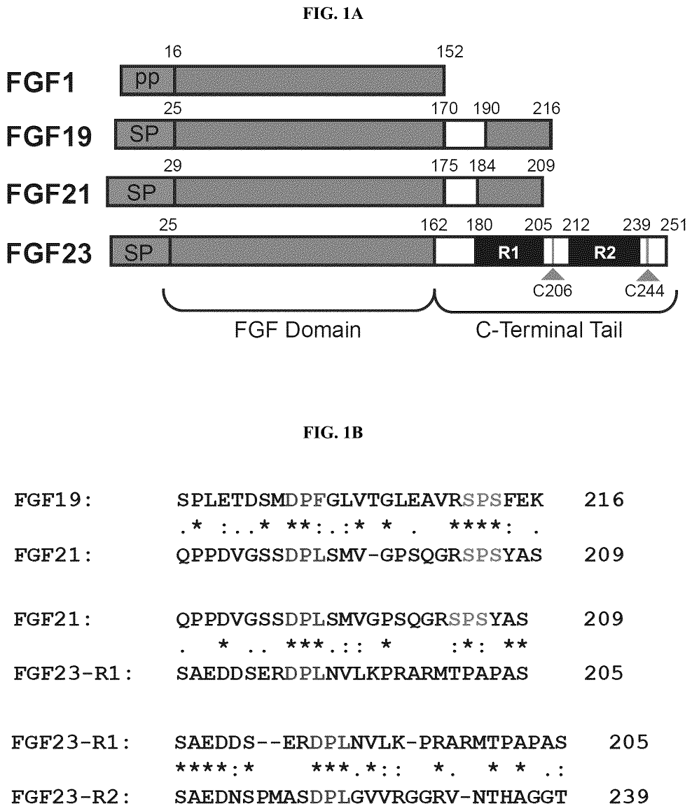

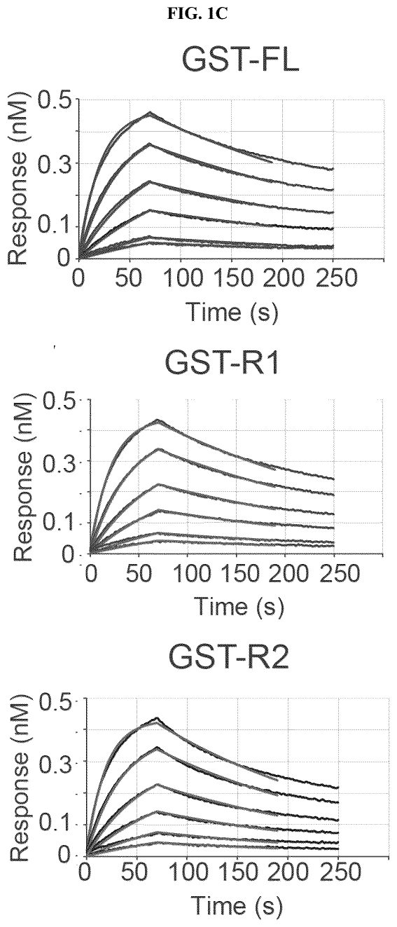

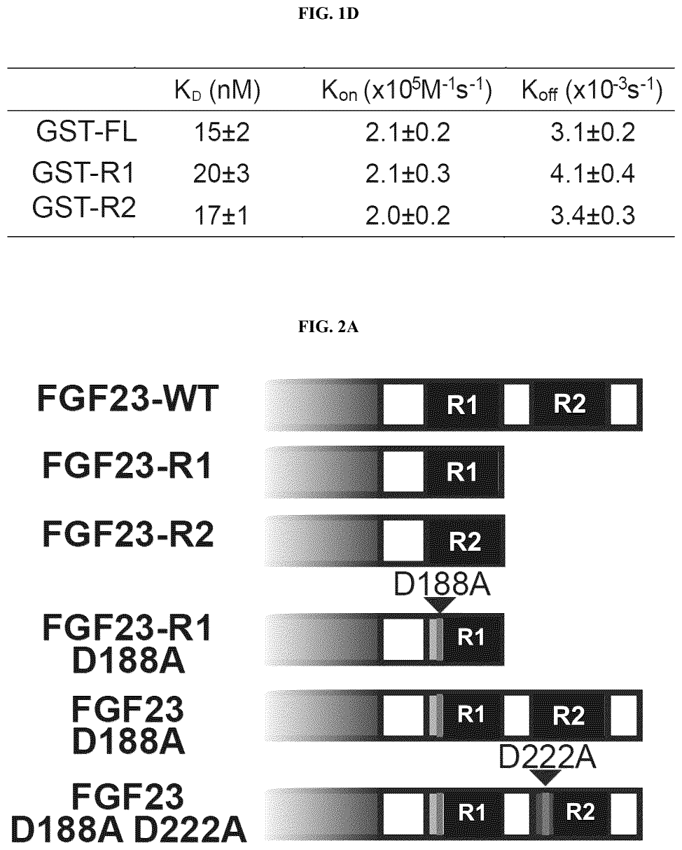

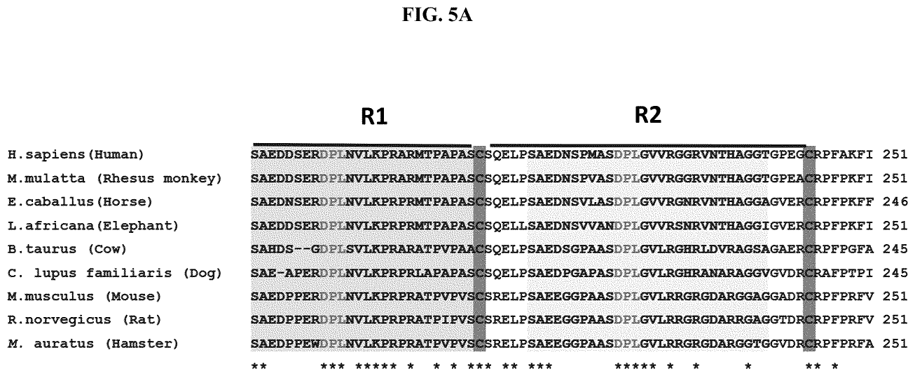

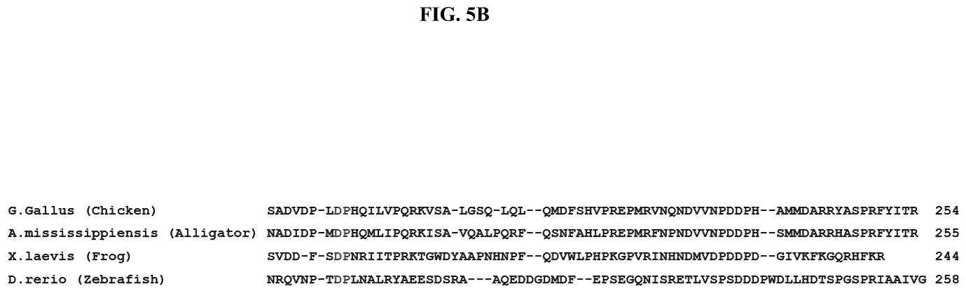

The crystal structures of the C-terminal tails (CTs) of FGF19 or FGF21 bound to the extracellular region β-Klotho (KLB) revealed conserved interactions along elongated interfaces that span both glycoside hydrolase-like domains D1 and D2 (also designated KL1 and KL2 domains) of β-Klotho (Olsen, et al., 2004, Proc Natl Acad Sci USA. 101:935-940; Kuzina, et al., 2019, Proc. Natl. Acad. Sci. U.S.A. 116:7819-7824). An FGF23 deletion mutant lacking 46 C-terminal amino acids was shown to be biologically active (Goetz, et al., 2010, Proc. Natl. Acad. Sci. U.S.A. 107:407-412). This deletion mutant was applied in the structural analysis of a complex containing the extracellular region of α-Klotho (sKLA) and FGFR1c extracellular domain that revealed conserved interactions primarily with D1 of sKLA (Chen, et al., 2018, Nature 553:461-466). Comparison of the primary structures of FGF19, FGF21 and FGF23 (FIG. 1A) shows that the C-terminal tails of FGF19 and 21 contain 46 and 34 amino acids, respectively while FGF23 contains a long C-terminal tail of 89 amino acids. Inspection of the primary structures shows that unlike FGF19 and FGF21, the C-terminal tail of FGF23 contains two homologous tandem repeats (FIG. 1B). Each repeat contains a DPL/F motif that is crucial for maintaining the compact and rigid structure necessary for binding to the D1 (KL1) site as well as a cluster of basic residues that bind to the D2 (KL2) site, indicating that a single FGF23 molecule may possess two separate binding regions for α-Klotho. It is noteworthy that, while all vertebrate FGF23 proteins have long C-terminal tails, only mammals have a second repeat homologous to the Klotho binding regions of FGF19,21 and 23. (FIG. 1B, FIG. 5B).

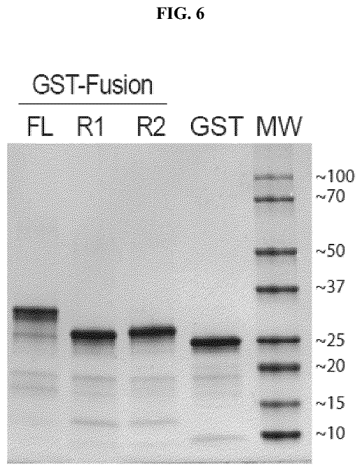

To examine the significance of each of the two FGF23 repeats in α-Klotho binding and receptor activation, bio-layer interferometry (BLI) analyses was applied to measure the kinetic parameters and dissociation constants of each repeat alone or the entire C-terminal tail of FGF23 towards sKLA. To that end, GST-fusion proteins expressing either full length (FL) tail of FGF23 (amino acids S1804251), the first repeat R1 (amino acids S180-S205), or the second repeat R2 (amino acids 5212-T239) were produced in E. coli (FIG. 6) and immobilized on BLI sensors. sKLA was produced in HEK293 EBNA cells and used as an analyte in the BLI measurements (see elsewhere herein).

The results from the BLI measurement (FIG. 1C) show that the GST fusion proteins with each single repeat or both repeats bind to sKLA with similar kinetic parameters and dissociation constants (Kd) of 15-20 nM (FIG. 1D), indicating that both R1 and R2 function as distinct bona fide ligands of α-Klotho and that FGF23-WT possesses two distinct binding sites for α-Klotho.

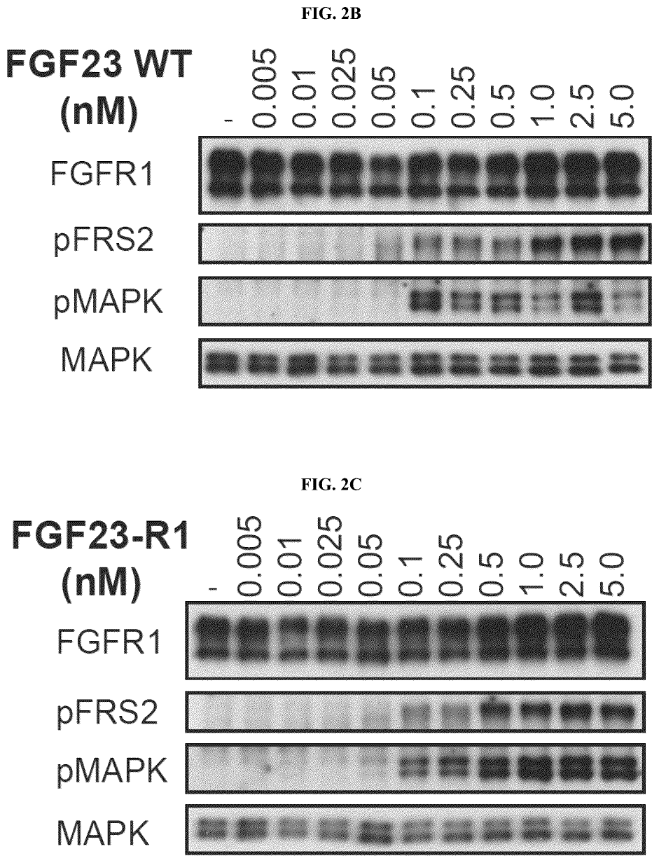

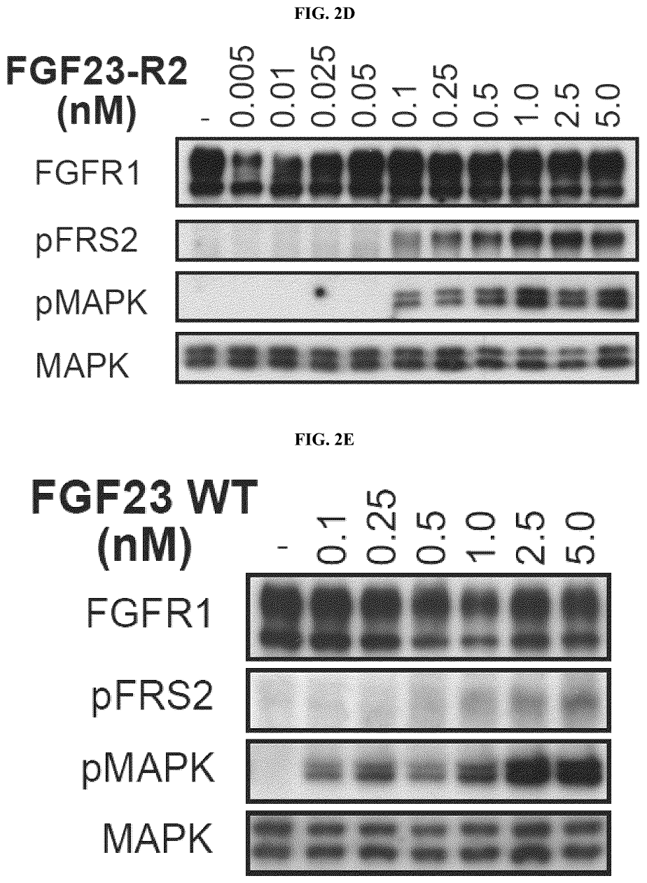

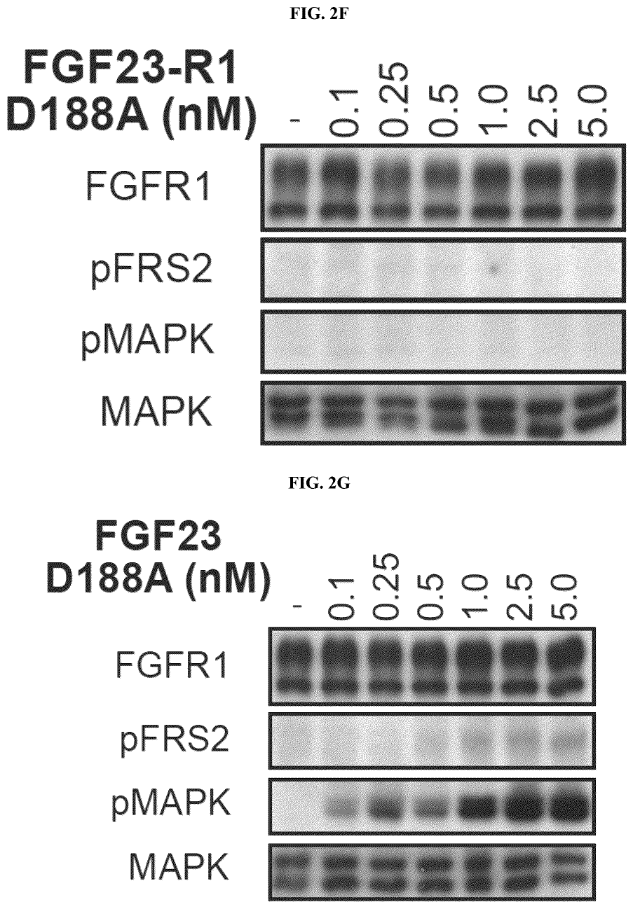

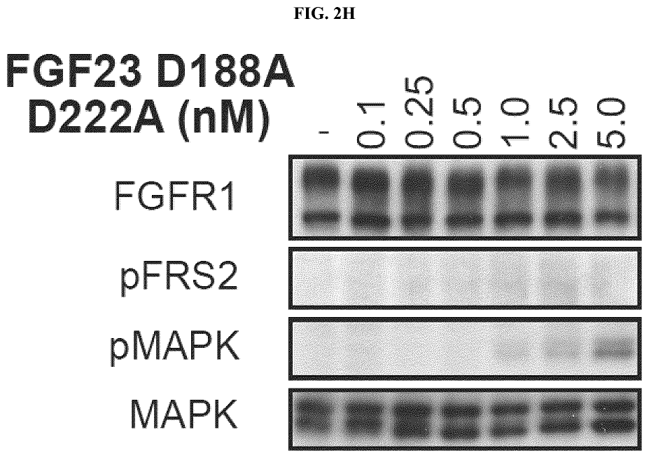

Since the BLI measurements clearly show that the FL tail of FGF23 as well as R1 and R2 form stable complexes with sKLA, next expressed and purified were FGF23 with the full length tail (FGF23-WT), FGF23 variants containing only one of the two repeats, FGF23-R1, and FGF23-R2, as well as FGF23 variants with one or both repeats inactivated by a point mutation in the DPL motif (D188A in R1 and D222A in R2) (FIG. 2A) and their ability to stimulate cell signaling was examined. HEK293 cells co-expressing FGFR1c and α-Klotho were stimulated with increasing concentrations of the different FGF23 variants (as indicated in FIGS. 2B-2H) for 10 minutes at 37° C. and lysates of unstimulated or ligand-stimulated cells were subjected to immunoblotting with anti-pFRS2α antibodies to monitor its phosphorylation as well as with anti-pMAPK antibodies and MAPK antibodies to monitor MAPK stimulation and MAPK expression, respectively.

The results presented in FIGS. 2B-2D show that FGF23-WT, FGF23-R1, and FGF23-R2 activate cell signaling to a similar extent as revealed by tyrosine phosphorylation of FRS2α and the activation of MAPK response (saturation is reached at 0.5-1.0 nM and at 0.1-nM ligand concentration respectively). By contrast, tyrosine phosphorylation of FRS2 and MAPK response were not detected in cells stimulated with FGF23-R1 D188A mutant (FIG. 2F). Interestingly, FGF23 D188A, a mutant with inactive R1 and a functional R2, stimulated tyrosine phosphorylation of FRS2α and activation of MAPK response to the same extent as FGF23-WT (FIG. 2G) indicating that FGF23 is capable of utilizing the second repeat (R2) alone, in the context of full-length C-terminal tail (separated by 50 amino acids from the FGF moiety) for α-Klotho binding and FGFR activation. Without wishing to be limited by any theory, this finding also raises questions whether the crystal structure of the ternary FGF23-R1/sKLA/FGFR1c complex may represent an oversimplified picture which does not depict the heterogeneity in the interactions between FGF23 and α-Klotho. Finally, in cells treated with FGF23 in which both R1 and R2 are inactivated by D188A/D222A double mutations, the tyrosine phosphorylation of FRS2α is completely abolished and MAPK activation is barely detectable (FIG. 2H).

### Example 2: R2 of FGF23 Functions as an Antagonist of FGF23-Induced Cell Signaling

The C-terminal region of FGF23 binds tightly to α-Klotho and, because it does not interact with FGFR, it may function as a competitor of FGF23 binding to α-Klotho and consequently as an inhibitor of FGF23 induced cell signaling. A full-length C-terminal peptide and a R1 peptide can antagonize FGF23 activation both in vitro and in vivo (Goetz, et al., 2010, Proc. Natl. Acad. Sci. U.S.A. 107:407-412; Agoro, et al., 2018, The FASEB Journal. 32:3752-3764).

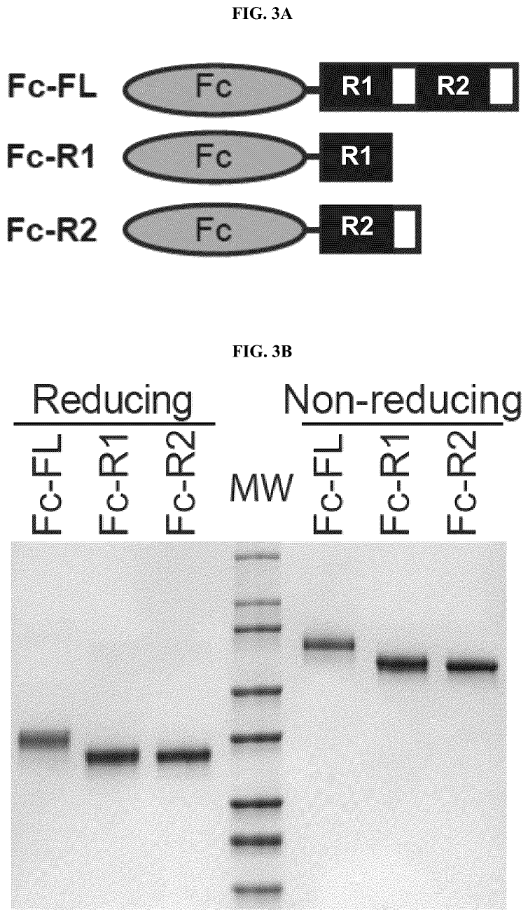

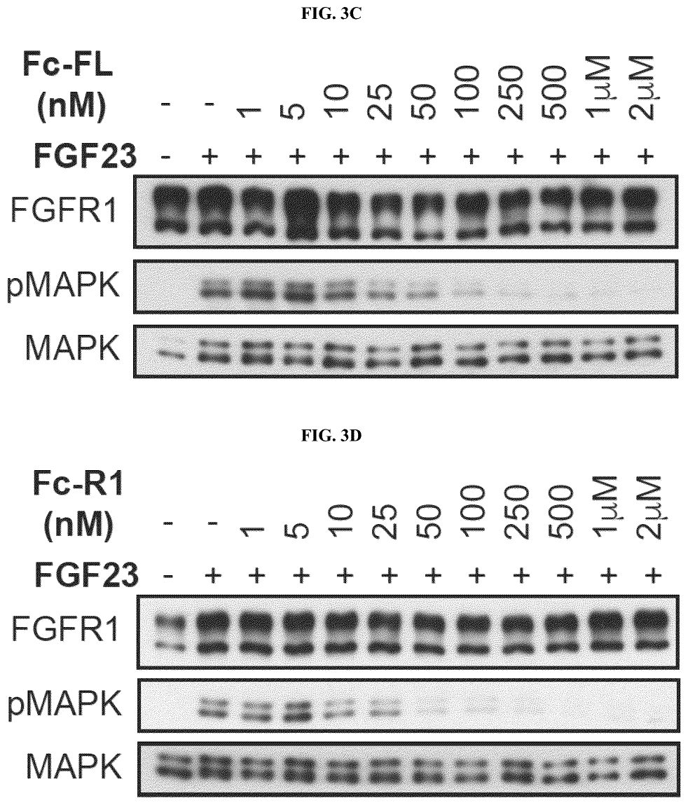

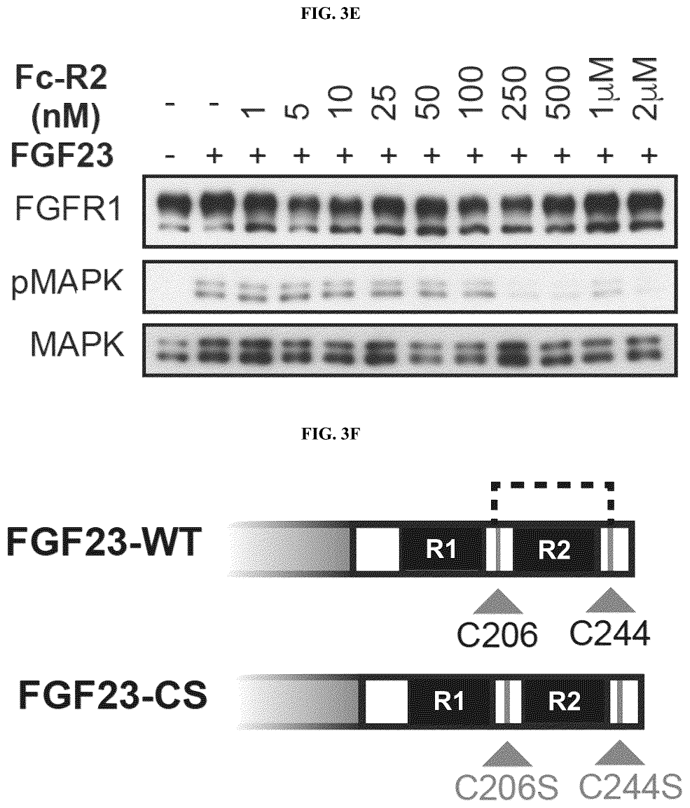

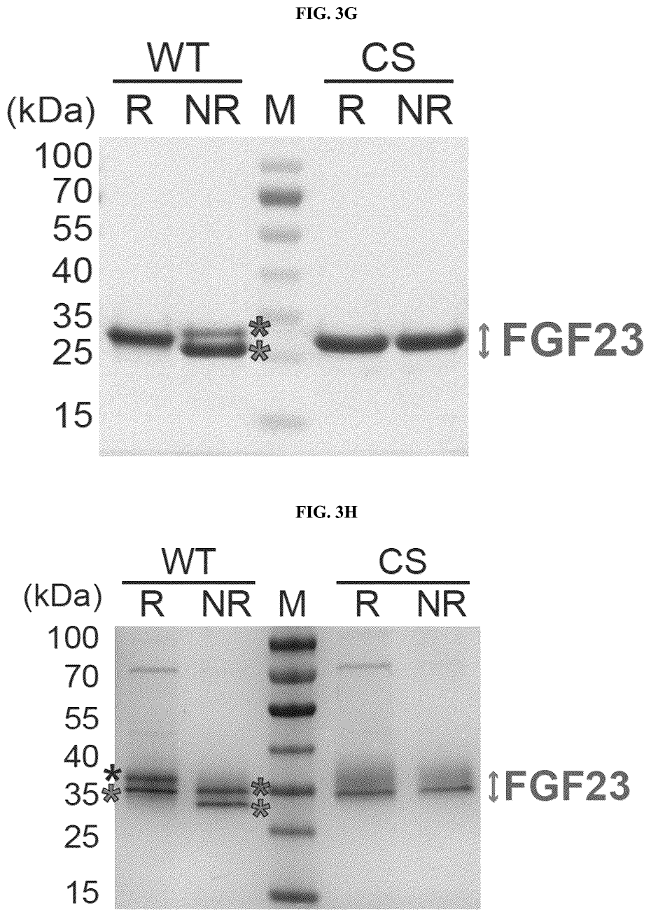

To test whether R2 (S212-T239) exerts similar antagonistic activity on FGF23 signaling and to compare its efficiency to those of full-length FGF23 C-tail (S1804251) or R1 peptide (S180-S205), these peptides were expressed and purified in a form of Fc fusion proteins, designated Fc-FL, Fc-R1 and Fc-R2 (FIGS. 3A-3B) and explored their effect upon FGF23-induced stimulation of HEK293 cells co-expressing α-Klotho and FGFR1c. Cells were incubated with increasing concentrations (as indicated, FIGS. 3C-3E) of individual Fc-fusion protein followed by stimulation with FGF23-WT for 10 minutes. Lysates from unstimulated or FGF23 stimulated cells were subjected to immunoblotting with anti-pMAPK antibodies to determine MAPK response or antibodies to FGFR1 and MAPK as controls for protein loading. The experiment presented in FIGS. 3C-3E shows that Fc-FL, Fc-R1 and Fc-R2 were able to completely inhibit FGF23-induced MAPK stimulation at similar concentrations (100-250 nM). These results demonstrate that Fc-R2 antagonizes FGF23-WT induced MAPK response similar to the antagonistic activities of Fc-R1 or Fc-FL tail. The ability of Fc-R2 to inhibit the formation of the αKlotho-FGFR signaling complex establishes it as a therapeutic for diseases resulting from increased FGF23 signaling.

### Example 3: Cysteine Residues Flanking R2 of FGF23 Form a Disulfide Bridge

Amino acid sequence alignments of FGF23 from different species (FIGS. 5A-5B) show that in mammals, the second repeat (R2) is flanked by two cysteine residues, e.g., Cys206 and Cys244, in human FGF23 (FIG. 3F). To determine whether these cysteine residues form a disulfide bridge, FGF23-WT was expressed in E. coli, and the refolded and purified protein was analyzed by SDS-PAGE under both reducing and non-reducing conditions in comparison to those of a mutant FGF23 in which both cysteines are substituted by serine residues (FGF23-CS).

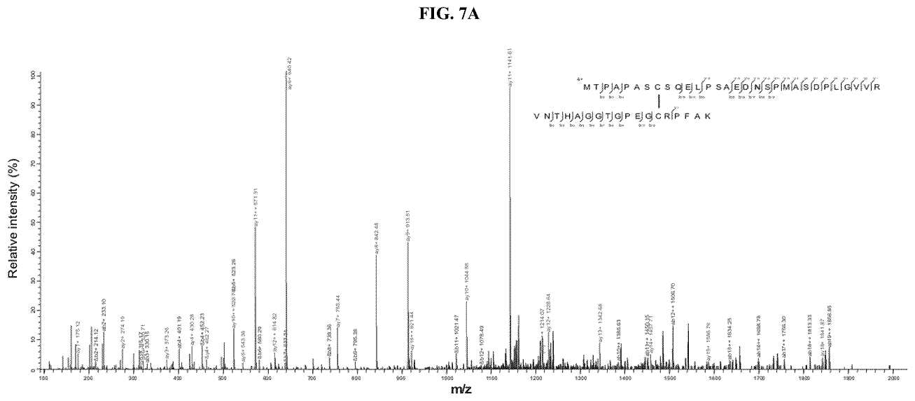

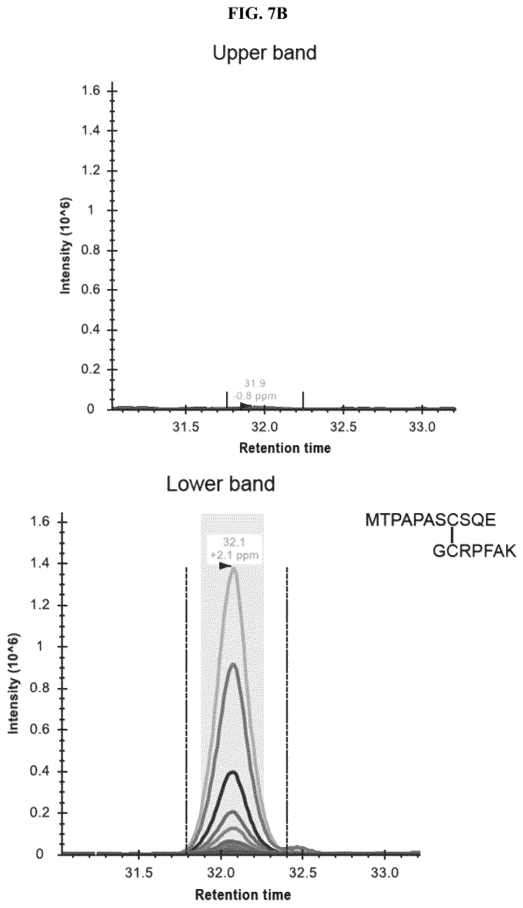

The experiment presented in FIG. 3G shows that FGF23-WT migrates on SDS-PAGE as a distinct single band under reduced (R) condition and as two bands (marked with two asterisks) under non-reducing condition (NR). The FGF23-CS mutant, on the other hand, migrates on SDS-PAGE as single distinct band under both reducing and non-reducing conditions. To determine if either of the two bands of FGF23-WT contains intramolecular disulfide bonds under non-reducing condition, each of the two bands were excised from the gel, subjected to trypsin and endoproteinase GluC digestion and analyzed by mass spectrometry to detect disulfide-linked peptides. The mass spectrometry analysis revealed that the lower band (FIG. 3G, lower asterisk, FIs. 7A-7B) contains peptides with intramolecular disulfide bond between Cys206 and Cys244. Only trace amounts of these peptides were detected in proteolytic digest of the upper band (FIG. 3G upper asterisk, FIG. 7B). While the majority of FGF23-WT expressed in bacteria becomes oxidized during refolding to form a disulfide bond between Cys206 and Cys244, the two cysteines are not bridged in a sub-population of refolded FGF23 molecules expressed in E. coli.

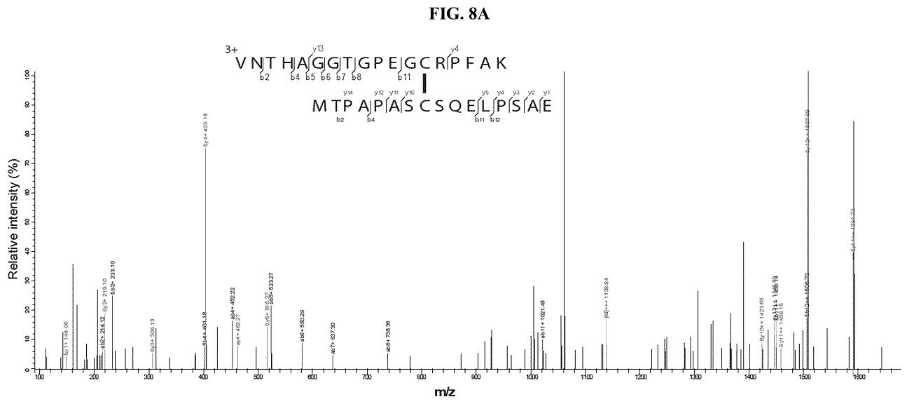

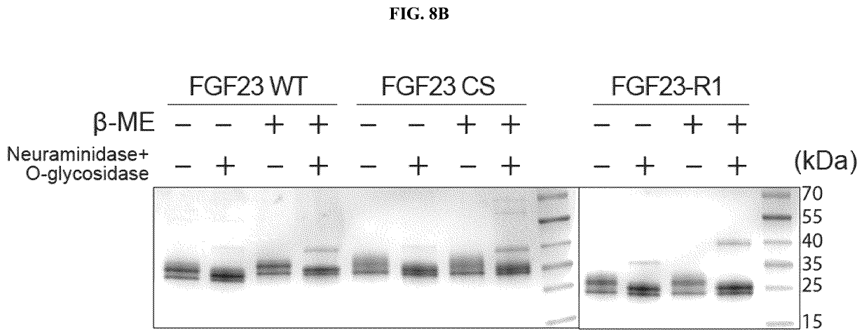

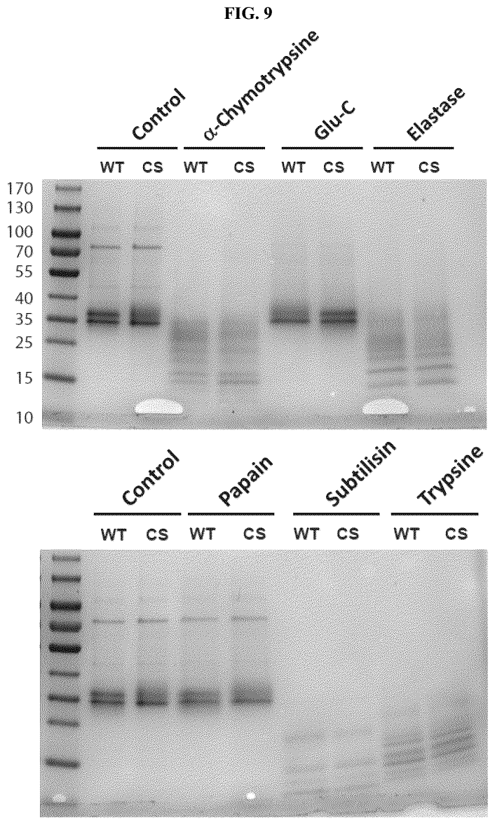

FLAG-tagged FGF23-WT and its CS mutant were next expressed in Expi293F cells (FIG. 3H) and both ligands were purified using affinity chromatography followed by size exclusion chromatography (se elsewhere herein). The SDS-PAGE analysis presented in FIG. 3H shows that FGF23-WT produced in mammalian cells (FGF23-WT) migrates as two distinct bands under both reducing and non-reducing conditions. Unlike bacterially expressed, FGF23 expressed in Expi293F cells is O-glycosylated and the two distinct bands visualized by SDS-PAGE under reducing and non-reducing conditions reveal different glycosylation forms of FGF23-WT which was confirmed by in vitro treatment with 0-glycosidase and α-(2→3,6,8,9)-Neuraminidase (FIG. 8B). FGF23-CS mutant expressed in Expi293 cells (FGF23-CS) migrated on SDS-PAGE as two distinct bands under both reducing and non-reducing conditions, due to differential glycosylation (FIGS. 8A-8B). The upper band of FGF23-CS is more smeared (FIG. 3H) than the corresponding band of FGF23-WT, suggesting potential heterogeneity in glycosylation patterns. Mass spectrometry analyses showed that FGF23 produced in Expi293F cells contained peptides with a disulfide bridge connecting Cys206 and Cys244. Without wishing to be limited by any theory, as it was proposed that O-linked glycosylation protects FGF23 from proteolysis, it was asked whether Cys206-Cys244 disulfide bridging affects FGF23 accessibility to proteolytic digestion. The experiment presented in FIG. 9 shows the results of limited proteolysis experiment with FGF23-WT and the CS mutant. Limited proteolysis of both proteins with various enzymes (as indicated) was performed using Proti-Ace Kit (Hampton Research) under the manufacturer's protocol. Digested samples were subjected to SDS-PAGE followed by Coomassie Blue staining to visualize the digested products. Based on the pattern of the bands as visualized by SDS-PAGE it was concluded that the protease-digested products of FGF23-WT and FGF23-CS are similar to each other and therefore cysteine Cys206-Cys244 disulfide bridging does not have a major impact on FGF23 accessibility to proteolytic digestion.

To explore the role of Cys206 to Cys244 disulfide formation on FGF23 binding to soluble α-Klotho, BLI measurements were used to compare the kinetic parameters and dissociation constants of FGF23-WT to those of FGF23-CS, FGF23 D188A, and FGF23-CS D188A expressed in Expi293F cells. The experiment presented in FIG. 10A show that all four FGF23 variants exhibit similar binding kinetics and dissociation constants towards sKLA in the range of 13 nM to 18 nM (Table 1). Furthermore, stimulation of HEK293 cells expressing α-Klotho and FGFR1c, with increasing concentrations of FGF23-WT or FGF23-CS revealed similar profile of tyrosine phosphorylation of FRS2a, MAPK response as well as similar serine phosphorylation of FGFR1c by activated MAPK, a feedback mechanism that leads to the attenuation of ligand stimulation (FIGS. 10B-10C). These results emphasize the ability of R2 in its oxidized form to create a ternary active complex with α-Klotho and FGFR1c.

TABLE 1
Binding of FGF23 variants to sKLA

| | K_{D} | Kₒₙ | K_{off} |
|---|---|---|---|
| | (nM) | (×10⁵ M⁻¹s⁻¹) | (×10⁻³ s⁻¹) |
| FGF23-WT | 14 ± 3 | 2.6 ± 0.5 | 3.4 ± 0.1 |
| FGF23-CS | 18 ± 4 | 2.3 ± 0.4 | 4.2 ± 0.4 |
| FGF23 D188A | 13 ± 3 | 2.8 ± 0.5 | 3.6 ± 0.2 |
| FGF23-CS D188A | 18 ± 3 | 2.5 ± 0.3 | 4.4 ± 0.3 |
| FGF23 D188A D222A* | n/a | n/a | n/a |

*no binding detected (no change in BLI signal)

### Example 4: FGF23 can Act as a Bivalent Ligand of α-Klotho Molecules Expressed on Cell Membrane

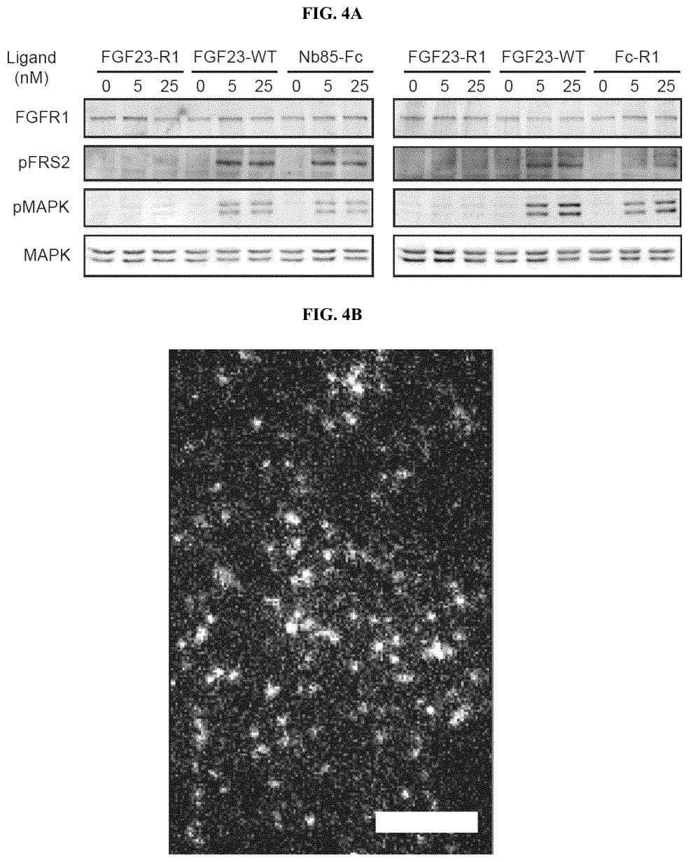

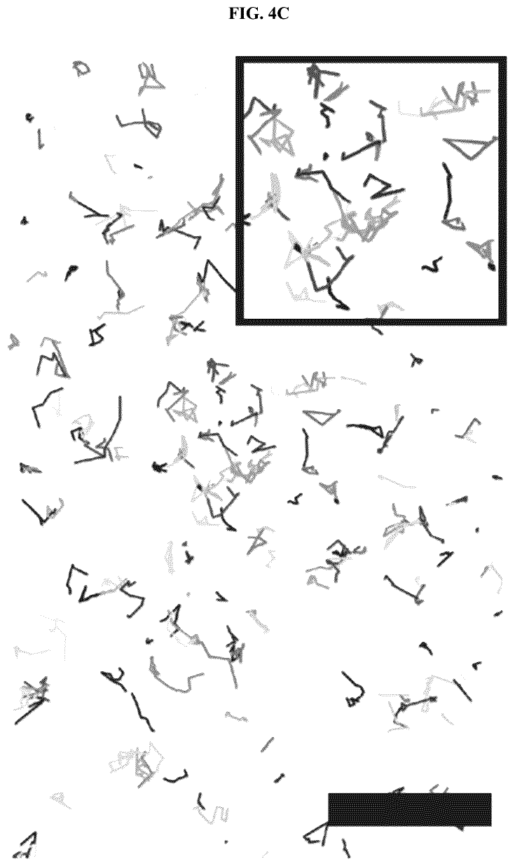

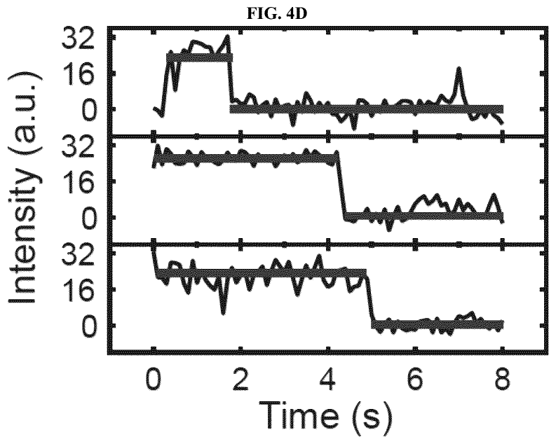

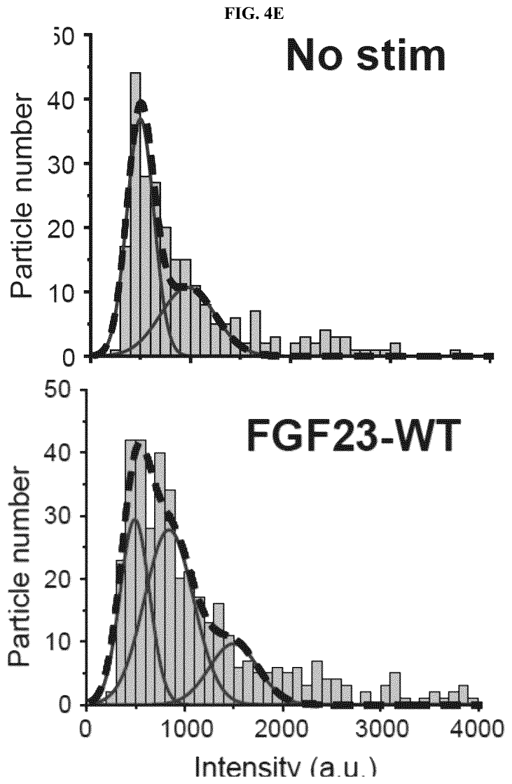

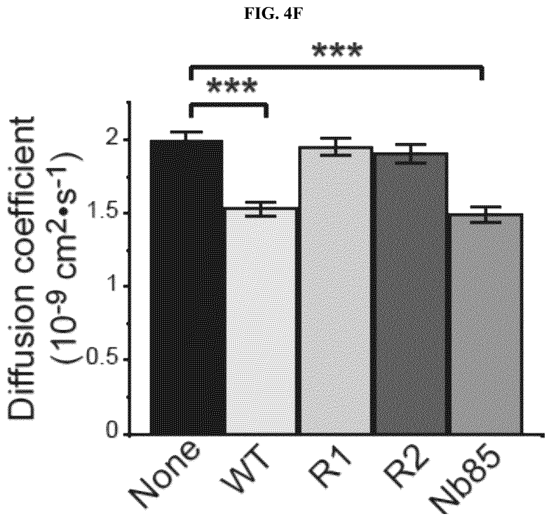

It was next examined whether a single FGF23-WT molecule is capable of binding via its R1 and R2 regions of the C-terminal tail to two α-Klotho molecules. In other words, the non-limiting aim of this experiment is to test the ability of FGF23-WT to function as a bivalent ligand of α-Klotho molecules located on the cell membrane. To address this question a chimeric receptor molecule composed of the extracellular domain of α-Klotho fused to the transmembrane and cytoplasmic domain of FGFR1 was constructed and expressed in L6 cells. In certain non-limiting embodiments, FGF23-WT may function as a bi-valent ligand capable of inducing dimerization of the chimeric receptor molecules, stimulating their tyrosine kinase activity and subsequent activation of downstream signaling. As positive controls, the activities of a dimeric Fc-nanobody that binds specifically to the extracellular domain of α-Klotho and a dimeric Fc-R1 fusion protein were analyzed for their ability to stimulate tyrosine phosphorylation of FRS2 and MAPK response in these cells. Cells expressing the chimeric α-Klotho-FGFR1c receptor were stimulated with 5 or 25 nM of FGF23-WT, FGF23-R1, the bivalent anti α-Klotho nanobody (Nb85-Fc) and Fc-R1 for 10 minutes at 37° C. Lysates from unstimulated or ligand stimulated cells were subjected to SDS-PAGE analysis followed by immunoblotting with anti-pFRS2α antibodies to monitor its phosphorylation, anti-pMAPK antibodies to monitor MAPK activation or anti-MAPK antibodies and anti-FGFR1 antibodies as control for protein loading. The experiment presented in FIG. 4D shows that both mammalian (left panel) and E. coli (right panel) produced FGF23-WT as well as bivalent α-Klotho nanobody and Fc-R1 protein induce robust activation of MAPK response. By contrast the monovalent FGF23-R1 variant (produced in E. coli or mammalian cells) failed to simulate MAPK response. These experiments demonstrate that FGF23-WT is capable of stimulating dimerization of α-Klotho molecules located on the cell membrane via its C-terminal tail (FIG. 10D).

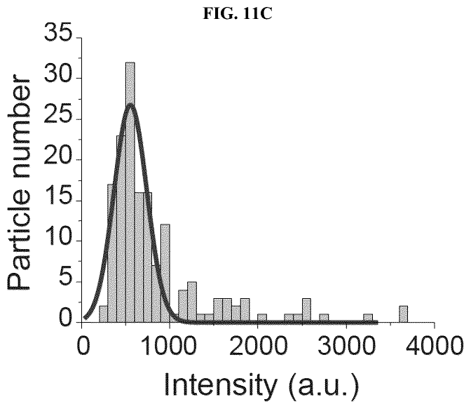

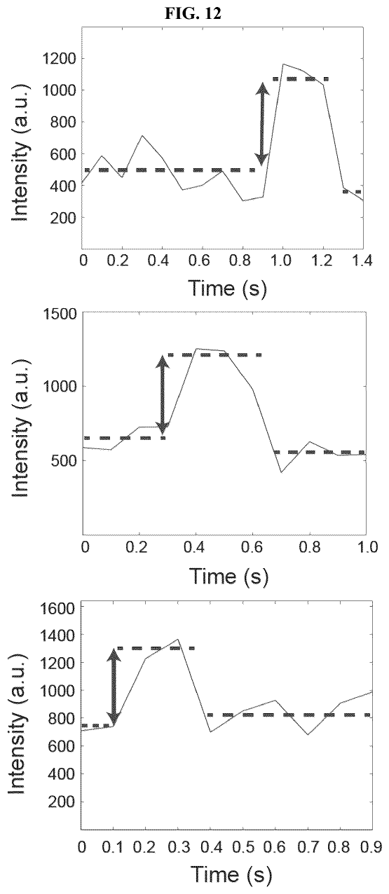

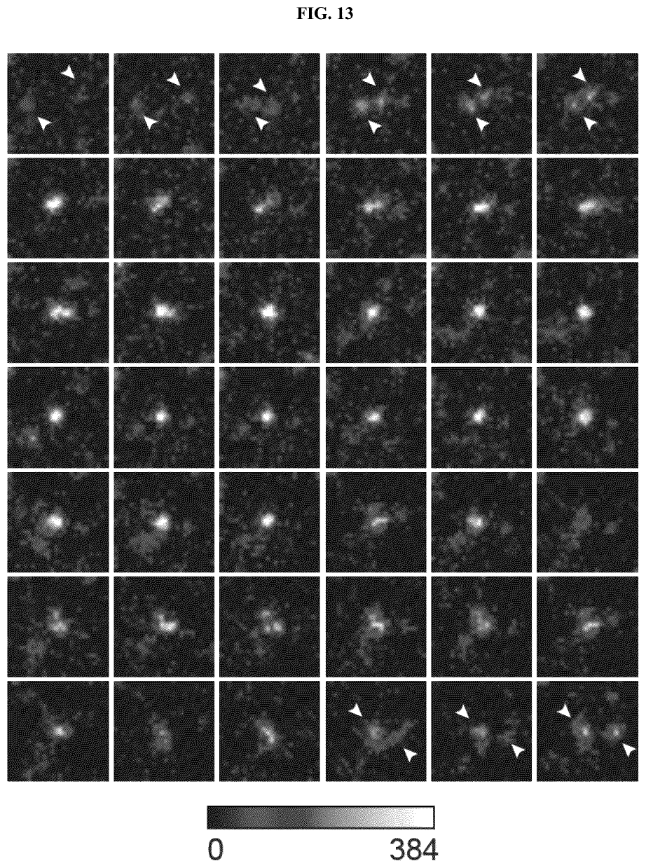

FGF23 stimulation of α-Klotho dimerization was next investigated using a single-molecule imaging approach. Visualization of α-Klotho molecules on the cell membrane was investigated by labeling α-Klotho fused to an N-terminal (extracellular) HaloTag with a cell-impermeant fluorescent HaloTag ligand Alexa488. L6 cells expressing low levels of HaloTag-α-Klotho were briefly labeled (15 min. at 37° C.) with Alexa488, and individual fluorescent particles were imaged using total internal reflection fluorescence (TIRF) microscopy to visualize individual α-Klotho molecules on the cell surface. FIG. 4B shows a representative TIRF microscopy image of a low expressing cell, with a particle density of particles/m 2, which is similar to the densities reported in single-molecule imaging studies of receptor dimerization (<0.45 particles/m 2). Particles were automatically detected and tracked to delineate their movements on the cell surface (FIG. 4C). Consistent with the particles representing single molecules, they often photobleached in a single step (FIG. 4D). The intensity distribution of particles in unstimulated cells could be fitted with a mixed Gaussian model, comprising a major peak with an intensity (498±16 a.u.) similar to that of free dye absorbed to glass (554±16 a.u.; FIGS. 11A-11C)— thus, likely corresponding to monomeric HaloTag-α-Klotho—and a minor peak with roughly twice the intensity (973±129 a.u). Without wishing to be limited by any theory, this second, smaller peak may reflect the dynamic equilibrium between monomers and dimers based on the intensity of individual tracks over time, which occasionally showed transient doubling (FIG. 12). Visual inspection of recordings also showed transient merging of particles (FIG. 13), although it is possible that these apparent merging events merely reflected the colocalization of particles, rather than their association, due to the diffraction limit of light. In contrast, when cells were stimulated with FGF23-WT, the intensity distribution became shifted to the right with the second (i.e. dimer) peak (840±57 a.u.) growing more prominent and a third peak forming with three times the monomer intensity (1502±89 vs. 492±23 a.u.). In addition to the quantized increase in particle intensity induced by FGF23-WT stimulation, the diffusion coefficients of the particles calculated from their mean square displacement (MSD) indicated that their diffusion coefficient (mean±SE=1.99±0.057×10⁻⁹ cm²·s⁻¹) was similarly reduced (by 22-23%, P<0.0001) by FGF23-WT binding (1.53±0.050×10⁻⁹ cm²·s⁻¹) as well as by binding of dimeric anti-α-Klotho nanobody Nb85-Fc (1.49±0.049×10⁻⁹ cm²·s⁻¹), but not by the monovalent FGF23-R1 or FGF23-R2 variants (1.95±0.059 and 1.91±0.065×10⁻⁹ cm²·s⁻¹, respectively). These results directly demonstrate that FGF23-WT acts as a bivalent ligand of α-Klotho molecules on the surface of living cells.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A non-natural soluble construct comprising an amino acid sequence that is at least 90% identical to amino acids 212-239 of SEQ ID NO:5 or a biologically active fragment thereof.

2. The construct of claim 1, which comprises amino acids 212-239 of SEQ ID NO:5 or a biologically active fragment thereof.

3. The construct of claim 2, which comprises amino acids 212-239 of SEQ ID NO:5.

4. The construct of claim 1, which is fused to a stability enhancing domain.

5. The construct of claim 4, wherein the stability enhancing domain comprises at least one of albumin, thioredoxin, glutathione S-transferase, and/or a Fc region of an antibody.

6. The construct of claim 5, wherein the Fc region is IgG Fc.

7. The construct of claim 6, wherein the Fc region is the Fc domain of human immunoglobulin 1 (IgG1), human immunoglobulin 2 (IgG2), human immunoglobulin 3 (IgG3), or human immunoglobulin 4 (IgG4).

8. The construct of claim 4, wherein the stability enhancing domain is fused with the N-terminus of the polypeptide or wherein the stability enhancing domain is fused with the C-terminus of the polypeptide.

9. (canceled)

10. The construct of claim 4, wherein the stability enhancing domain is directly fused to the polypeptide or wherein the stability enhancing domain is fused through a linker to the polypeptide.

11. (canceled)

12. The constrict of claim 10, wherein the linker comprises about 1-18 amino acids or 1-20 (independently selected ethylene glycol or propylene glycol) units.

13. The construct of claim 10,
wherein the C-terminus of the linker fused to the N-terminus of the polypeptide is not one of the following: APASCSQELP (SEQ ID NO:20), PASCSQELP (SEQ ID NO:21), ASCSQELP (SEQ ID NO:22), SCSQELP (SEQ ID NO:23), CSQELP (SEQ ID NO:24), SQELP (SEQ ID NO:25), QELP (SEQ ID NO:26), ELP, LP, P, or
wherein the N-terminus of the linker fused to the C-terminus of the polypeptide is not one of the following: GPEGCRPFAKF (SEQ ID NO:27), GPEGCRPFAK (SEQ ID NO:28), GPEGCRPFA (SEQ ID NO:29), GPEGCRPF (SEQ ID NO:30), GPEGCRP (SEQ ID NO:31), GPEGCR (SEQ ID NO:32), GPEGC (SEQ ID NO:33), GPEG (SEQ ID NO:34), GPE, GP, G.

14. (canceled)

15. The construct of claim 1, which is pegylated, at least partially methylated, or C-terminus amidated.

16. A nucleic acid sequence that encodes the construct of claim 1.

17. A vector comprising the nucleic acid sequence of claim 16, optionally wherein the vector is an expression vector.

18. (canceled)

19. The vector of claim 17, which is an autonomously replicating or an integrative mammalian cell vector.

20. A cell, cells, or a plurality of cells comprising the nucleic acid of claim 16.

21. A method of treating, ameliorating, or preventing an endocrine FGF-related disease or disorder in a mammal, the method comprising administering to the mammal a therapeutically effective amount of the construct of claim 1.

22. The method of claim 21, wherein at least one of the following applies:
(a) the construct prevents or minimizes binding of FGF23 to α-Klotho on the surface of the mammal's cell;
(b) the disease or disorder includes hypophosphatemia and/or tumor-induced osteomalacia;
(c) the mammal is human;
(d) the construct is administered by an administration route selected from the group consisting of inhalational, oral, rectal, vaginal, parenteral, intracranial, topical, transdermal, pulmonary, intranasal, buccal, ophthalmic, intrathecal, and intravenous;
(e) the construct or a precursor thereof is delivered on an encoded vector, wherein the vector encodes the construct or precursor thereof and, upon administration of the vector to the subject, the construct is transcribed and translated from the vector;
(f) the mammal is further administered at least one additional drug that treats or prevents the disease and/or disorder.

23-27. (canceled)

28. The method of claim 22, wherein if (f) the construct and the at least one additional drug are co-administered, optionally wherein the construct and the at least one additional drug are co-formulated.

29. (canceled)


SEQUENCE LISTING (34 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - protein, 1044 aa, Homo sapiens
MKPGCAAGSPGNEWIFFSTDEITTRYRNTMSNGGLQRSVILSALILLRAVTGFSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 4 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 5 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 6 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 618 aa, Homo sapiens
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 8 - protein, 72 aa, Macaca mulatta
SAEDDSERDPLNVLKPRARMTPAPASCSQELPSAEDNSPVASDPLGVVRGGRVNTHAGGTGPEACRPFPKFI

SEQ ID NO: 9 - protein, 72 aa, Equus caballus
SAEDNSERDPLNVLKPRPRMTPAPASCSQELPSAEDNSVLASDPLGVVRGNRVNTHAGGAGVERCRPFPKFF

SEQ ID NO: 10 - protein, 72 aa, Loxodonta africana
SAEDDSERDPLNVLKPRPRMTPAPASCSQELLSAEDNSVVANDPLGVVRSNRVNTHAGGIGVERCRPFPKFI

SEQ ID NO: 11 - protein, 70 aa, Bos taurus
SAHDSGDPLSVLKPRARATPVPAACSQELPSAEDSGPAASDPLGVLRGHRLDVRAGSAGAERCRPFPGFA

SEQ ID NO: 12 - protein, 71 aa, Canis familiaris
SAEAPERDPLNVLKPRPRLAPAPASCSQELPSAEDPGAPASDPLGVLRGHRANARAGGVGVDRCRAFPTPI

SEQ ID NO: 13 - protein, 72 aa, Mus musculus
SAEDPPERDPLNVLKPRPRATPVPVSCSRELPSAEEGGPAASDPLGVLRRGRGDARGGAGGADRCRPFPRFV

SEQ ID NO: 14 - protein, 72 aa, Rattus norvegicus
SAEDPPERDPLNVLKPRPRATPIPVSCSRELPSAEEGGPAASDPLGVLRRGRGDARRGAGGTDRCRPFPRFV

SEQ ID NO: 15 - protein, 72 aa, Mesocricetus auratus
SAEDPPEWDPLNVLKPRPRATPVPVSCSRELPSAEEGGPAASDPLGVLRRGRGDARGGTGGVDRCRPFPRFA

SEQ ID NO: 16 - protein, 71 aa, Gallus gallus
SADVDPLDPHQILVPQRKVSALGSQLQLQMDFSHVPREPMRVNQNDVVNPDDPHAMMDARRYASPRFYITR

SEQ ID NO: 17 - protein, 72 aa, Alligator mississippiensis
NADIDPMDPHQMLIPQRKISAVQALPQRFQSNFAHLPREPMRFNPNDVVNPDDPHSMMDARRHASPRFYITR

SEQ ID NO: 18 - protein, 68 aa, Xenopus laevis
SVDDFSDPNRIITPRKTGWDYAAPNHNPFQDVWLPHPKGPVRINHNDMVDPDDPDGIVKFKGQRHFKR

SEQ ID NO: 19 - protein, 73 aa, Danio rerio
NRQVNPTDPLNALRYAEESDSRAAQEDDGDMDFEPSEGQNISRETLVSPSDDDPWDLLHDTSPGSPRIAAIVG

SEQ ID NO: 20 - protein, 10 aa, Artificial Sequence
  Chemically synthesized
APASCSQELP

SEQ ID NO: 21 - protein, 9 aa, Artificial Sequence
  Chemically synthesized
PASCSQELP

SEQ ID NO: 22 - protein, 8 aa, Artificial Sequence
  Chemically synthesized
ASCSQELP

SEQ ID NO: 23 - protein, 7 aa, Artificial Sequence
  Chemically synthesized
SCSQELP

SEQ ID NO: 24 - protein, 6 aa, Artificial Sequence
  Chemically synthesized
CSQELP

SEQ ID NO: 25 - protein, 5 aa, Artificial Sequence
  Chemically synthesized
SQELP

SEQ ID NO: 26 - protein, 4 aa, Artificial Sequence
  Chemically synthesized
QELP

SEQ ID NO: 27 - protein, 11 aa, Artificial Sequence
  Chemically synthesized
GPEGCRPFAKF

SEQ ID NO: 28 - protein, 10 aa, Artificial Sequence
  Chemically synthesized
GPEGCRPFAK

SEQ ID NO: 29 - protein, 9 aa, Artificial Sequence
  Chemically synthesized
GPEGCRPFA

SEQ ID NO: 30 - protein, 8 aa, Artificial Sequence
  Chemically synthesized
GPEGCRPF

SEQ ID NO: 31 - protein, 7 aa, Artificial Sequence
  Chemically synthesized
GPEGCRP

SEQ ID NO: 32 - protein, 6 aa, Artificial Sequence
  Chemically synthesized
GPEGCR

SEQ ID NO: 33 - protein, 5 aa, Artificial Sequence
  Chemically synthesized
GPEGC

SEQ ID NO: 34 - protein, 4 aa, Artificial Sequence
  Chemically synthesized
GPEG